(19) United States
(12) Patent Application Publication
(10) Pub. No.: US 2021/0309962 A1
(43) Pub. Date: Oct. 7, 2021

(54) COMPOSITIONS AND METHODS UTILIZING AMNIOTIC FLUID STEM CELLS

(71) Applicants: Bruce K. Young, New York, NY (US); Michael K. Chan, Brooklyn, NY (US)

(72) Inventors: Bruce K. Young, New York, NY (US); Michael K. Chan, Brooklyn, NY (US)

(21) Appl. No.: 17/219,371

(22) Filed: Mar. 31, 2021

Related U.S. Application Data
(60) Provisional application No. 63/003,569, filed on Apr. 1, 2020.

(51) Int. Cl. C12N 5/073; A61K 35/50

(52) U.S. Cl. CPC C12N 5/0605; A61K 35/50

(57) ABSTRACT

Amniotic fluid stem cell clones are provided. Also provided are pharmaceutical compositions comprising the amniotic fluid stem cell clones, methods of generating the amniotic fluid stem cell clones, and methods of using the amniotic fluid stem cell clones, including for treating subjects and for bioassays.

### CROSS REFERENCE

This application claims the benefit of U.S. Provisional Patent Application No. 63/003,569, filed Apr. 1, 2020, which is incorporated herein by reference in its entirety.

### BACKGROUND

Stem cells have great potential for therapies in a wide range of conditions. Bone marrow, peripheral blood cells, umbilical cord cells and embryonic tissue have been examined as potential sources of stem cells in human therapies, as have adult cells restored to pluripotency by genetic manipulation. Several problems exist with the use of these cells. In particular, these cells are limited in number, are difficult to maintain in culture and expand, and are prone to induce tumorigenesis.

### INCORPORATION BY REFERENCE

Each patent, publication, and non-patent literature cited in the application is hereby incorporated by reference in its entirety as if each was incorporated by reference individually.

### SUMMARY

Disclosed herein, in some embodiments, is a composition comprising a population of amniotic fluid stem cells, wherein at least 65% of cells in the population of amniotic fluid stem cells are CD90 positive, wherein the population of amniotic fluid stem cells is in a unit dosage form, wherein the population comprises at least 50 cells.

Disclosed herein, in some embodiments, is a pharmaceutical composition comprising a population of amniotic fluid stem cells and a pharmaceutically-acceptable excipient, wherein at least 65% of cells in the population of amniotic fluid stem cells are CD90 positive.

Disclosed herein, in some embodiments, is a composition comprising a population of amniotic fluid stem cells, wherein at least 90% of cells in the population of amniotic fluid stem cells are CD90 positive, wherein at least 90% of cells in the population of amniotic fluid stem cells remain CD90 positive after two weeks in culture.

Disclosed herein, in some embodiments, is a composition comprising a clonal population of amniotic fluid stem cells, wherein the clonal population of amniotic fluid stem cells has been prepared by a process comprising: (a) isolating a single CD90 positive amniotic fluid stem cell from a plurality of amniotic fluid stem cells based on expression of CD90; and (b) expanding the single CD90 positive amniotic fluid stem cell to generate the clonal population of amniotic fluid stem cells.

Disclosed herein, in some embodiments, is a composition comprising a population of amniotic fluid stem cells, wherein the population of amniotic fluid stem cells has been prepared by a process comprising: (a) sorting a population of cells comprising amniotic fluid stem cells to obtain an enriched population of amniotic fluid stem cells comprising at least 90% CD90 positive cells; (b) expanding the enriched population of amniotic fluid stem cells in culture to obtain an expanded population of amniotic fluid stem cells; (c) sorting the expanded population of amniotic fluid stem cells to obtain a second enriched population of amniotic fluid stem cells comprising at least 90% CD90 positive cells; and (d) expanding the second enriched population of amniotic fluid stem cells in culture to obtain a second expanded population of amniotic fluid stem cells.

Disclosed herein, in some embodiments, is a method of generating a clonal population of amniotic fluid stem cells, comprising: (a) isolating a single CD90 positive amniotic fluid stem cell from a plurality of cells based on expression of CD90; and (b) expanding the single CD90 positive amniotic fluid stem cell to generate the clonal population of amniotic fluid stem cells.

Disclosed herein, in some embodiments, is a method of generating a population of amniotic fluid stem cells, comprising: (a) sorting a population of cells comprising amniotic fluid stem cells to obtain an enriched population of amniotic fluid stem cells comprising at least 90% CD90 positive cells; (b) expanding the enriched population of amniotic fluid stem cells in culture to obtain an expanded population of amniotic fluid stem cells; (c) sorting the expanded population of amniotic fluid stem cells to obtain a second enriched population of amniotic fluid stem cells comprising at least 90% CD90 positive cells; and (d) expanding the second enriched population of amniotic fluid stem cells in culture to obtain a second expanded population of amniotic fluid stem cells.

Disclosed herein, in some embodiments, is a method of determining an effect of an agent on stem cells and/or fetal cells, comprising contacting a population of cells with the agent, wherein the population of cells comprise amniotic fluid stem cells, and determining the effect of the agent on the amniotic fluid stem cells, wherein at least 65% of the cells in the population of cells are CD90 positive, wherein the population of cells comprises at least 50 cells.

Disclosed herein, in some embodiments, is a method of determining an effect of an agent on stem cells, comprising: (a) isolating a single CD90 positive amniotic fluid stem cell from a plurality of cells; (b) expanding the single CD90 positive amniotic fluid stem cell to generate a clonal population of amniotic fluid stem cells; (c) contacting the clonal population of amniotic fluid stem cells with the agent; and (d) determining the effect of the agent on the clonal population of amniotic fluid stem cells.

Disclosed herein, in some embodiments, is a method of determining an effect of an agent on stem cells, comprising: (a) sorting a population of cells comprising amniotic fluid stem cells to obtain an enriched population of amniotic fluid stem cells comprising at least 90% CD90 positive cells; (b) expanding the enriched population of amniotic fluid stem cells in culture to obtain an expanded population of amniotic fluid stem cells; (c) sorting the expanded population of amniotic fluid stem cells to obtain a second enriched population of amniotic fluid stem cells comprising at least 90% CD90 positive cells; (d) expanding the second enriched population of amniotic fluid stem cells in culture to obtain a second expanded population of amniotic fluid stem cells; (e) contacting the second expanded population of amniotic fluid stem cells with the agent; and (f) determining the effect of the agent on the second expanded population of amniotic fluid stem cells.

### BRIEF DESCRIPTION OF THE FIGURES

FIG. 1 shows a skin graft from a BALB/c mouse (white) onto a wound area of a C57BL/6 mouse (black). The graft was treated with clonal amniotic fluid stem cells of the disclosure. The graft appears healthy and hair is growing on the graft treated with clonal amniotic fluid stem cells, indicating enhanced vascularization.

FIG. 2A, FIG. 2B, and FIG. 2C show that amniotic fluid stem cell clones differentiate towards a neuronal lineage upon culture in neural induction medium. FIG. 2A illustrates mean fluorescence intensity of Nestin. FIG. 2B illustrates mean fluorescence intensity of Beta-Tubulin III. FIG. 2C illustrates mean fluorescence intensity of glial fibrillary acidic protein (GFAP).

FIG. 3 shows that amniotic fluid stem cell clones differentiate towards a chondrogenic lineage upon culture in chondrogenic induction medium, as indicated by mean fluorescence intensity of Aggrecan.

FIG. 4A and FIG. 4B show that amniotic fluid stem cell clones differentiate down a definitive endoderm lineage. FIG. 4A illustrates expression of SOX17. FIG. 4B illustrates expression of CXCR4.

FIG. 5 illustrates expression of stem cell transcription factors by an amniotic fluid stem cell cone of the disclosure.

### DETAILED DESCRIPTION

Stem cells represent a promising source of potential therapies for a wide range of conditions. Stem cells can be undifferentiated cells capable of prolonged self-replication without differentiation, and can be characterized by the presence of surface markers and transcription factors. Stem cells can also be characterized by their ability to differentiate into functional cells of various cell lineages from multiple germ layers (endoderm, mesoderm and ectoderm). Some stem cells can give rise to tissues of multiple germ layers following transplantation. Based on the developmental potential, stem cells can be classified as: totipotent stem cells, which can have the potential to differentiate into lineages from mesodermal, ectodermal and endodermal tissues, such as osteogenic, neurogenic and hepatic lineages; pluripotent stem cells, which can have the potential to differentiate into various embryonic cell types; multipotent stem cells, which can have the potential to differentiate into cell lineages all within a particular tissue, organ, or physiological system; oligopotent stem cells, which can have the potential to differentiate into a few cell lineages; and unipotent stem cells, which can have the potential to differentiate into a single cell lineage.

The utility of stem cells from many sources is limited by factors such as availability, limited numbers, difficulty maintaining in culture, and propensity for tumorigenesis. Amniotic fluid stem cells represent a source of stem cells with the potential to overcome these limitations.

Amniotic Fluid Stem Cells

Amniotic fluid can be a protective liquid contained in the amniotic sac and surrounding the developing fetus in the uterus. Amniotic fluid can provide mechanical protection and can facilitate the exchange of nutrients required for fetal growth and health. The composition of amniotic fluid can change with gestational age. Amniotic fluid can contain significant quantities of cells derived from embryonic tissues, such as the skin and the respiratory, urinary and gastrointestinal tracts, and pluripotent stem cells. Amniotic fluid cells can be a valuable source of multipotential stem cells and in some embodiments are not subject to the problems encountered with the use of other stem cells.

Amniotic fluid stem cells can be obtained from amniocentesis, for example, obtained from second trimester pregnancies undergoing genetic amniocentesis. The cells can be grown and multiplied, can be non-tumorigenic, and can be routinely sampled and grown in tissue culture for genetic testing. However, amniotic fluid stem cells can be highly heterogeneous and subject to extensive individual variations that can limit utility in therapeutics and research.

In some embodiments, the disclosure provides clonal amniotic fluid stem cell lines that are derived from a single cell, maintain stemness throughout time in culture, and can be effectively used to develop drug bioassays and potential therapies. Disclosed herein are amniotic fluid stem cell clones that are useful for regenerative medicine and drug bioassays. Amniotic fluid stem cells disclosed herein can exhibit reduced heterogeneity compared to stem cells freshly isolated from amniotic fluid. Amniotic fluid stem cells disclosed herein can be clonal. For example, amniotic fluid stem cells with reduced heterogeneity can be generated by expanding a single cell into a larger population of cells.

Amniotic fluid stem cells disclosed herein can exhibit a high self-renewal capacity and plasticity. Amniotic fluid stem cells disclosed herein can maintain expression of one or more markers disclosed herein even after being cultured for relatively long periods. For example, in some embodiments, a population of amniotic fluid stem cells disclosed herein can maintain expression of a marker disclosed herein for at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 90, at least about 100, at least about 110, at least about 120, at least about 130, at least about 140, at least about 150, at least about 160, at least about 170, at least about 180, at least about 190, at least about 200, at least about 225, at least about 250, or at least about 300 passages or doublings. In some embodiments a population of amniotic fluid stem cells disclosed herein can maintain expression of a marker disclosed herein for at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 weeks in culture. Maintaining expression of a marker can comprise maintaining a percentage of cells in a population that are positive for a marker as disclosed herein, for example, maintaining at least 65%, at least 90%, or at least 95% of cells in a population as CD90 positive. Maintaining expression of a marker can comprise maintaining a level of expression of a marker in a population, e.g., maintaining an expression level of a marker as determined by mean fluorescence intensity or quantitative real time PCR. In some embodiments, amniotic fluid stem cells disclosed herein can maintain a stable karyotype. In some embodiments, amniotic fluid stem cells disclosed herein can maintain an undifferentiated phenotype.

Differentiation can be a process by which an unspecialized cell acquires the features of a specialized cell such as a heart, liver, lung, pancreas, muscle, or neuronal cell. When a cell differentiates into a mesodermal, ectodermal or endodermal lineage, the cell can become committed to a specific mesodermal, ectodermal or endodermal lineage, respectively. Non-limiting examples of cells that differentiate into a mesodermal lineage or give rise to specific mesodermal cells can include cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal cells. Non-limiting examples of cells that differentiate into an ectodermal lineage or give rise to specific ectodermal cells can include cells that are epidermal cells, neurogenic cells, and neurogliagenic cells. Non-limiting examples of cells that differentiate into an endodermal lineage or give rise to specific endodermal cells can include cells that are alveolargenic, epatogenic, and pancreatogenic.

In some embodiments, amniotic fluid stem cells disclosed herein have the ability to integrate into and regenerate damaged tissue thanks to their multi-lineage differentiation capacity. Moreover, the disclosed human amniotic fluid clonal stem cells have the ability to attach and proliferate on biodegradable scaffolds, and can be used for surgical implantation both in utero and after birth. Therefore, the disclosed human amniotic fluid stem cells disclosed herein can be useful for treating or correcting congenital malformations of the fetus, and diseases associated with damaged tissues or dysfunctional organs in human subjects.

In some embodiments, at least about at least about 10%, at least about 20%, at least about 30%, at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% of cells in a composition, a population of cells, or a population of amniotic fluid stem cells can be viable.

Cell Markers

A cell or a population of cells of the disclosure can be characterized by the expression of one or more markers disclosed herein. A marker can be or comprise, for example, a nucleic acid, polypeptide, a lipid (e.g., a glycosphingolipids), or a carbohydrate that is differentially expressed by a cell of interest, such that the cell of interest can be identified and distinguished from other cells. Non-limiting examples of markers found on stem cells include, SSEA3, SSEA4, Tra-1-60, Tra-1-81, CD117 and CD90.

Cells that express a marker can be referred to as ‘positive’ for that marker. Cells that do not express a marker can be referred to as being ‘negative’ for that marker. ‘Positive’ and ‘negative’ can also be applied relative to other cells rather than simply representing a presence or absence. For example, in some embodiments, ‘positive’ can refer to cells expressing a relatively higher level of a marker as compared to other cells. In some embodiments, ‘negative’ can refer to cells that express a relatively lower level of a marker as compared to other cells. A level of expression (e.g., a percentage of cells the express the marker above a threshold, or an average level of expression in a population of cells) can be determined using suitable methods, for example, flow cytometry, mass spectrometry, ELISA, Western Blot, RNA-seq, qPCR, etc.

In some embodiments, a population of cells of the disclosure is characterized by the percentage of cells that express CD90, SSEA4, TRA-1-60, CD9, CD15, CD29, CD44, CD73, CD105, CD117, CD133, Nanog, Oct-4, Rex-1, Sox-2, or a combination thereof. In some embodiments, a population of cells of the disclosure is characterized by the percentage of cells that express CD90. In some embodiments, a population of cells of the disclosure is characterized by the percentage of cells that express CD90 and the percentage of cells that express SSEA4. In some embodiments, a population of cells of the disclosure is characterized by the percentage of cells that express CD90 and the percentage of cells that express TRA-1-60. In some embodiments, a population of cells of the disclosure is characterized by the percentage of cells that express CD90, the percentage of cells that express SSEA4. and the percentage of cells that express TRA-1-60.

In some embodiments, a population of cells of the disclosure is characterized by the percentage of cells that co-express two or more markers selected from the group consisting of CD90, SSEA4, TRA-1-60, CD9, CD15, CD29, CD44, CD73, CD105, CD117, CD133, Nanog, Oct-4, Rex-1, and Sox-2. In some embodiments, a population of cells of the disclosure is characterized by the percentage of cells that co-express CD90 and SSEA4. In some embodiments, a population of cells of the disclosure is characterized by the percentage of cells that co-express CD90 and TRA-1-60. In some embodiments, a population of cells of the disclosure is characterized by the percentage of cells that co-express CD90, SSEA4, and TRA-1-60.

A composition of the disclosure can comprise cells that are CD9 positive. CD9 can be a cell-surface glycoprotein member of the transmembrane 4 superfamily, also known as the tetraspanin family, characterized by the presence of four hydrophobic domains. Tetraspanins can be cell surface glycoproteins with four transmembrane domains that form multimeric complexes with other cell surface proteins and are involved in many cellular processes including differentiation, adhesion, and signal transduction.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD9 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD9 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD9 positive.

A composition of the disclosure can comprise cells that are CD15 positive. CD15 can be a carbohydrate adhesion molecule, also known as 3-fucosyl-N-acetyl-lactosamine and SSEA1 (stage-specific embryonic antigen 1). CD15 can be a marker for pluripotent stem cells and can mediate phagocytosis and chemotaxis.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD15 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD15 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD15 positive.

A composition of the disclosure can comprise cells that are CD29 positive. CD29 can be a cell surface receptor, also known as Integrin beta-1 (ITGB1), which in humans is encoded by the ITGB gene. This integrin can associate with integrins alpha 1 and 2 to form integrin complexes which function as collagen receptors.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD29 positive. In some embodiments, at least 90% of cells in a composition, a population of cells, or a population of amniotic fluid stem cells can be CD29 positive. In some embodiments, at least 95% of cells in a composition, a population of cells, or a population of amniotic fluid stem cells can be CD29 positive. In some embodiments, at least 97% of cells in a composition, a population of cells, or a population of amniotic fluid stem cells can be CD29 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD29 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD29 positive. In some embodiments, about 90% to about 99.9% of cells in a composition, a population of cells, or a population of amniotic fluid stem cells can be CD29 positive. In some embodiments, about 95% to about 99.9% of cells in a composition, a population of cells, or a population of amniotic fluid stem cells can be CD29 positive.

A composition of the disclosure can comprise cells that are CD44 positive. CD44 can be a cell-surface glycoprotein involved in cell-cell interactions, cell adhesion and migration. CD44 can be a receptor for hyaluronic acid (HA) and can also interact with other ligands, such as osteopontin, collagens, and matrix metalloproteinases (MMPs).

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD44 positive. In some embodiments, at least 60% of cells in a composition, a population of cells, or a population of amniotic fluid stem cells can be CD44 positive. In some embodiments, at least 90% of cells in a composition, a population of cells, or a population of amniotic fluid stem cells can be CD44 positive. In some embodiments, at least 95% of cells in a composition, a population of cells, or a population of amniotic fluid stem cells can be CD44 positive. In some embodiments, at least 98% of cells in a composition, a population of cells, or a population of amniotic fluid stem cells can be CD44 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD44 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD44 positive. In some embodiments 90% to 100% of cells in a composition, a population of cells, or a population of amniotic fluid stem cells can be CD44 positive.

A composition of the disclosure can comprise cells that are CD73 positive. CD73 can be a glycosyl-phosphatidylinositol (GPI)-linked 70-kDa cell surface enzyme encoded by the NTSE gene, also known as ecto-5′-nucleotidase.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD73 positive. In some embodiments, at least about 95% of cells in a composition, a population of cells, or a population of amniotic fluid stem cells can be CD73 positive. In some embodiments, at least about 98% of cells in a composition, a population of cells, or a population of amniotic fluid stem cells can be CD73 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD73 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD73 positive.

A composition of the disclosure can comprise cells that are CD90 positive. CD90 can be a 25-37 kDa glycophosphatidylinositol (GPI)-linked glycoprotein, also known as Thy1, expressed by stem cells, endothelial cells, hematopoietic stem cells and neurogenic cells.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD90 positive. In some embodiments, at least 65% of cells in a composition, a population of cells, or a population of amniotic fluid stem cells can be CD90 positive. In some embodiments, at least 80% of cells in a composition, a population of cells, or a population of amniotic fluid stem cells can be CD90 positive. In some embodiments, at least 90% of cells in a composition, a population of cells, or a population of amniotic fluid stem cells can be CD90 positive. In some embodiments, at least 95% of cells in a composition, a population of cells, or a population of amniotic fluid stem cells can be CD90 positive. In some embodiments, at least 97% of cells in a composition, a population of cells, or a population of amniotic fluid stem cells can be CD90 positive. In some embodiments, at least 99% of cells in a composition, a population of cells, or a population of amniotic fluid stem cells can be CD90 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD90 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD90 positive. In some embodiments, 65-99.9% of cells in a composition, a population of cells, or a population of amniotic fluid stem cells can be CD90 positive. In some embodiments, 90-99.9% of cells in a composition, a population of cells, or a population of amniotic fluid stem cells can be CD90 positive. In some embodiments, 95-99.9% of cells in a composition, a population of cells, or a population of amniotic fluid stem cells can be CD90 positive.

A composition of the disclosure can comprise cells that are CD105 positive. CD105 can be vascular endothelium glycoprotein that plays an important role in the regulation of angiogenesis. CD105 can contribute to normal structure and integrity of vasculature. CD105 can contribute to normal extraembryonic angiogenesis and for embryonic heart development.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD105 positive. In some embodiments, at least 5% of cells in a composition, a population of cells, or a population of amniotic fluid stem cells can be CD105 positive. In some embodiments, at least 10% of cells in a composition, a population of cells, or a population of amniotic fluid stem cells can be CD105 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD105 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD105 positive.

A composition of the disclosure can comprise cells that are CD117 positive. CD117 can be a receptor tyrosine kinase protein, also known as proto-oncogene c-Kit or tyrosine-protein kinase Kit, which in humans is encoded by the KIT gene and is expressed on the surface of hematopoietic stem cells.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD117 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD117 positive. In some embodiments, at most 20% of cells in a composition, a population of cells, or a population of amniotic fluid stem cells can be CD117 positive. In some embodiments, at most 10% of cells in a composition, a population of cells, or a population of amniotic fluid stem cells can be CD117 positive. In some embodiments, at most 5% of cells in a composition, a population of cells, or a population of amniotic fluid stem cells can be CD117 positive. In some embodiments, at most 3% of cells in a composition, a population of cells, or a population of amniotic fluid stem cells can be CD117 positive. In some embodiments, no CD117 positive cells are present in a composition, a population of cells, or a population of amniotic fluid stem cells.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD117 positive.

A composition of the disclosure can comprise cells that are CD133 positive. CD133 can be an antigen, also known as prominin-1, which in humans is encoded by the PROM1 gene and is expressed on the surface of hematopoietic stem cells.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be CD133 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be CD133 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be CD133 positive.

A composition of the disclosure can comprise cells that are SSEA3 positive or negative.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be SSEA3 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be SSEA3 positive. In some embodiments at most 20% of cells in a composition, a population of cells, or a population of amniotic fluid stem cells can be SSEA3 positive. In some embodiments at most 10% of cells in a composition, a population of cells, or a population of amniotic fluid stem cells can be SSEA3 positive. In some embodiments at most 5% of cells in a composition, a population of cells, or a population of amniotic fluid stem cells can be SSEA3 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be SSEA3 positive.

A composition of the disclosure can comprise cells that are SSEA4 positive. SSEA4 (stage-specific embryonic antigen-4) can be a glycolipid carbohydrate present on undifferentiated pluripotent stem cells. Expression of human SSEA4 can decrease following differentiation of human embryonic carcinoma cells, but can increase following differentiation in mouse.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be SSEA4 positive. In some embodiments, at least about 10% of cells in a composition, a population of cells, or a population of amniotic fluid stem cells can be SSEA4 positive. In some embodiments, at least about 25% of cells in a composition, a population of cells, or a population of amniotic fluid stem cells can be SSEA4 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be SSEA4 positive. In some embodiments, at most 50% of cells in a composition, a population of cells, or a population of amniotic fluid stem cells can be SSEA4 positive. In some embodiments, at most 20% of cells in a composition, a population of cells, or a population of amniotic fluid stem cells can be SSEA4 positive. In some embodiments, at most 10% of cells in a composition, a population of cells, or a population of amniotic fluid stem cells can be SSEA4 positive. In some embodiments, at most 5% of cells in a composition, a population of cells, or a population of amniotic fluid stem cells can be SSEA4 positive. In some embodiments, at most 1% of cells in a composition, a population of cells, or a population of amniotic fluid stem cells can be SSEA4 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be SSEA4 positive. In some embodiments, 10-50% of cells in a composition, a population of cells, or a population of amniotic fluid stem cells can be SSEA4 positive. In some embodiments, 25-30% of cells in a composition, a population of cells, or a population of amniotic fluid stem cells can be SSEA4 positive. In some embodiments, 25-30% of cells in a composition, a population of cells, or a population of amniotic fluid stem cells can be SSEA4 positive. In some embodiments, 0-20% of cells in a composition, a population of cells, or a population of amniotic fluid stem cells can be SSEA4 positive. In some embodiments, 0-10% of cells in a composition, a population of cells, or a population of amniotic fluid stem cells can be SSEA4 positive.

A composition of the disclosure can comprise cells that are TRA-1-60 positive. TRA-1-60 can be a >200 kDa pluripotent stem cell-specific protein expressed on the surface of undifferentiated human embryonic stem (ES) and induced pluripotent stem (iPS) cells, embryonal carcinoma (EC) cells, and embryonic germ (EG) cells, as well as rhesus monkey ES cell lines. The epitope, which can be lost upon cell differentiation, can contains sialic acid and can be associated with a large-molecular-mass transmembrane protein named podocalyxin.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be TRA-1-60 positive. In some embodiments, at least 5% of cells in a composition, a population of cells, or a population of amniotic fluid stem cells can be TRA-1-60 positive. In some embodiments, at least 3% of cells in a composition, a population of cells, or a population of amniotic fluid stem cells can be TRA-1-60 positive. In some embodiments, at least 1% of cells in a composition, a population of cells, or a population of amniotic fluid stem cells can be TRA-1-60 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be TRA-1-60 positive. In some embodiments, at most 30% of cells in a composition, a population of cells, or a population of amniotic fluid stem cells can be TRA-1-60 positive. In some embodiments, at most 10% of cells in a composition, a population of cells, or a population of amniotic fluid stem cells can be TRA-1-60 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be TRA-1-60 positive. In some embodiments, 5-10% of cells in a composition, a population of cells, or a population of amniotic fluid stem cells can be TRA-1-60 positive. In some embodiments, 3-30% of cells in a composition, a population of cells, or a population of amniotic fluid stem cells can be TRA-1-60 positive. In some embodiments, 3-15% of cells in a composition, a population of cells, or a population of amniotic fluid stem cells can be TRA-1-60 positive.

A composition of the disclosure can comprise cells are Nanog positive. Nanog can be a homeodomain-containing transcription factor essential for maintenance of pluripotency and self-renewal in embryonic stem cells. Expression of Nanog can be controlled by a network of factors including Sox2 and Oct-4.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be Nanog positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be Nanog positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be Nanog positive.

In some embodiments, a population of cells or a population of amniotic fluid stem cells of the disclosure expresses a level of Nanog that is within 1.5-fold, within 2-fold, within 3-fold, within 4-fold, within 5-fold, within 6-fold, within 7-fold, within 8-fold, within 9-fold, within 10-fold, within 15-fold, within 20-fold, within 25-fold, within 30-fold, within 40-fold, within 50-fold, within 100-fold, within 200-fold, within 300-fold, within 400-fold, within 500-fold, within 600-fold, within 700-fold, within 800-fold, within 900-fold, within 1000-fold, or within 10,000-fold of the level of expression in CD90-positive cells that have been freshly isolated from amniotic fluid.

A composition of the disclosure can comprise cells that are OCT-4 positive. OCT-4 can be a transcription factor expressed in undifferentiated pluripotent embryonic stem cells and germ cells during normal development. Together with Sox2 and Nanog, OCT-4 can contribute to the maintenance of pluripotent potential.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be OCT-4 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be OCT-4 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be OCT-4 positive.

In some embodiments, a population of cells or a population of amniotic fluid stem cells of the disclosure expresses a level of OCT-4 that is within 1.5-fold, within 2-fold, within 3-fold, within 4-fold, within 5-fold, within 6-fold, within 7-fold, within 8-fold, within 9-fold, within 10-fold, within 15-fold, within 20-fold, within 25-fold, within 30-fold, within 40-fold, within 50-fold, within 100-fold, within 200-fold, within 300-fold, within 400-fold, within 500-fold, within 600-fold, within 700-fold, within 800-fold, within 900-fold, within 1000-fold, or within 10,000-fold of the level of expression in CD90-positive cells that have been freshly isolated from amniotic fluid.

A composition of the disclosure can comprise cells that are SOX-2 positive. SOX-2, also referred to as SRY (sex determining region Y)-box 2, can be a transcription factor expressed in undifferentiated pluripotent embryonic stem cells and germ cells during development. Together with Oct-4 and Nanog, SOX-2 can contribute to the maintenance of pluripotent potential.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be SOX-2 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be SOX-2 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be SOX-2 positive.

In some embodiments, a population of cells or a population of amniotic fluid stem cells of the disclosure expresses a level of SOX-2 that is within 1.5-fold, within 2-fold, within 3-fold, within 4-fold, within 5-fold, within 6-fold, within 7-fold, within 8-fold, within 9-fold, within 10-fold, within 15-fold, within 20-fold, within 25-fold, within 30-fold, within 40-fold, within 50-fold, within 100-fold, within 200-fold, within 300-fold, within 400-fold, within 500-fold, within 600-fold, within 700-fold, within 800-fold, within 900-fold, within 1000-fold, or within 10,000-fold of the level of expression in CD90-positive cells that have been freshly isolated from amniotic fluid.

A composition of the disclosure can comprise cells that are REX-1 positive. REX-1 can be a transcription factor that is involved in stem cell self-renewal. REX-1 can be a marker for pluripotency. REX-1 can be involved in the reprogramming of X-chromosome inactivation during the acquisition of pluripotency.

For example, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, or at least about 99.99% of cells in a composition, a population of cells, or a population of amniotic fluid stem cells can be REX-1 positive.

In some embodiments, at most about 1%, at most about 2%, at most about 3%, at most about 4%, at most about 5%, at most about 6%, at most about 7%, at most about 8%, at most about 9%, at most about 10%, at most about 11%, at most about 12%, at most about 13%, at most about 14%, at most about 15%, at most about 16%, at most about 17%, at most about 18%, at most about 19%, at most about 20%, at most about 21%, at most about 22%, at most about 23%, at most about 24%, at most about 25%, at most about 26%, at most about 27%, at most about 28%, at most about 29%, at most about 30%, at most about 31%, at most about 32%, at most about 33%, at most about 34%, at most about 35%, at most about 36%, at most about 37%, at most about 38%, at most about 39%, at most about 40%, at most about 41%, at most about 42%, at most about 43%, at most about 44%, at most about 45%, at most about 46%, at most about 47%, at most about 48%, at most about 49%, at most about 50%, at most about 51%, at most about 52%, at most about 53%, at most about 54%, at most about 55%, at most about 56%, at most about 57%, at most about 58%, at most about 59%, at most about 60%, at most about 61%, at most about 62%, at most about 63%, at most about 64%, at most about 65%, at most about 66%, at most about 67%, at most about 68%, at most about 69%, at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% of cells in a composition, a population of cells, or a population of amniotic fluid stem cells can be REX-1 positive.

In some embodiments, about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 0% to about 20%, about 0% to about 30%, about 0% to about 40%, about 0% to about 50%, about 0% to about 60%, about 0% to about 70%, about 0% to about 80%, about 0% to about 90%, about 10% to about 30%, about 10% to about 40%, about 10% to about 50%, about 10% to about 60%, about 10% to about 70%, about 10% to about 80%, about 10% to about 90%, about 10% to about 100%, about 20% to about 40%, about 20% to about 50%, about 20% to about 60%, about 20% to about 70%, about 20% to about 80%, about 20% to about 90%, about 20% to about 100%, about 30% to about 50%, about 30% to about 60%, about 30% to about 70%, about 30% to about 80%, about 30% to about 90%, about 30% to about 100%, about 40% to about 60%, about 40% to about 70%, about 40% to about 80%, about 40% to about 90%, about 40% to about 100%, about 50% to about 70%, about 50% to about 80%, about 50% to about 90%, about 50% to about 100%, about 60% to about 80%, about 60% to about 90%, about 60% to about 100%, about 70% to about 90%, about 70% to about 100%, or about 80% to about 100% of cells in a composition, a population of cells, or a population of amniotic fluid stem cells can be REX-1 positive.

In some embodiments, a population of cells or a population of amniotic fluid stem cells of the disclosure expresses a level of REX-1 that is within 1.5-fold, within 2-fold, within 3-fold, within 4-fold, within 5-fold, within 6-fold, within 7-fold, within 8-fold, within 9-fold, within 10-fold, within 15-fold, within 20-fold, within 25-fold, within 30-fold, within 40-fold, within 50-fold, within 100-fold, within 200-fold, within 300-fold, within 400-fold, within 500-fold, within 600-fold, within 700-fold, within 800-fold, within 900-fold, within 1000-fold, or within 10,000-fold of the level of expression in CD90-positive cells that have been freshly isolated from amniotic fluid.

Pharmaceutical Compositions and Methods of Administration

Disclosed herein, in some embodiments, is a pharmaceutical composition comprising a population of amniotic fluid stem cells. The pharmaceutical composition can comprise a clonal population of amniotic fluid stem cells disclosed herein (e.g., a population expanded from a single cell). The pharmaceutical composition can comprise two or more a clonal populations of amniotic fluid stem cells disclosed herein (e.g., populations expanded from separate single cells).

A pharmaceutical composition disclosed herein can be a combination of amniotic fluid stem cells described herein with other chemical components, such as carriers, stabilizers, diluents, dispersing agents, suspending agents, thickening agents, and/or excipients. The pharmaceutical composition can improve the stability of the cells and can facilitate administration of the cells to a subject.

A composition of the disclosure can comprise buffers such as neutral buffered saline or phosphate buffered saline; carbohydrates such as glucose, mannose, sucrose or dextrans; mannitol; proteins; polypeptides or amino acids such as glycine; antioxidants; chelating agents such as EDTA or glutathione; preservatives, or a combination thereof. Compositions of the present invention can be formulated for intravenous administration (e.g., intravenous injection or infusion). A composition of the disclosure can be a sterile liquid preparation, for example, an isotonic aqueous solution, suspension, emulsion, dispersion, or viscous composition. In some embodiments, an excipient that can be used is saline. In some embodiments, an excipient that can be used is phosphate buffered saline (PBS).

A composition of the disclosure can be buffered to a selected pH. For example a composition of the disclosure can be buffered to a pH of 5-9, 5-8, 5-7, 5-6, 6-9, 6-8, 6-7, 7-9, 7-8, 6.5-8.5, 6.5-8, 6.5-7.7, 6.5-7.6, 6.5-7.5, 6.5-7.4, 6.5-7.3, 6.5-7.2, 6.5-7, 7-7.7, 7-7.6, 7-7.5, 7-7.4, 7-7.3, 7-7.2, 7-7.1, 7.2-7.6, 7.2-7.5, 7.2-7.4, 7.3-7.7, 7.3-7.6, 7.3-7.5, 7.34-7.45, 7.0-7.2, 7.2-7.4, 7.3-7.5, 7.4-7.6, or 7.6-7.8. A composition can comprise a pH buffer, such as 0.1 mM-100 mM phosphate pH 6.0-9.0, 0.1-100 mM HEPES pH 6.0-9.0, 0.1 mM-100 mM bicarbonate pH 6.0-9.0, 0.1 mM-100 mM citrate pH 6.0-9.0, 0.1-100 mM acetate pH 4.0-8.0, or any combination thereof.

The composition can comprise electrolytes, such as 5 mM-400 mM NaCl, 0.5 mM-50 mM KCl, 0.05 mM-50 mM CaCl₂, 0.05 mM-50 mM MgCl₂, 0.05 mM-50 mM LiCl₂, 0.05 mM-50 mM MnCl₂, or any combination thereof.

The composition can comprise an anti-oxidant, such as glutathione (reduced), glutathione (oxidized), β-mercaptoethanol, dithiothreitol, ascorbate, tris(2-carboxyethyl)phosphine, or any combination thereof.

The composition can comprise a stabilizer, such as 0.01%-10% human serum albumin, 0.01%-10% bovine serum albumin, 0.1%-99% human serum, 0.1%-99% fetal bovine serum, 0.01%-10% IgG, 0.1%-10% immunoglobin, 0.06%-60% trehalose, or molecular polymers such as 0.1%-20% polyethylene glycol (MW 200-20,000,000), or any combination thereof.

Liquid or viscous compositions can comprise carriers, which can be a solvent or dispersing medium containing, for example, water, saline, phosphate buffered saline, polyol (for example, glycerol, propylene glycol, or liquid polyethylene glycol) and suitable mixtures thereof.

The compositions can be isotonic, i.e., having a substantially similar osmotic pressure as blood. The isotonicity of a composition herein can be adjusted using sodium chloride, or other pharmaceutically-acceptable agents such as dextrose, boric acid, sodium tartrate, propylene glycol, or other inorganic or organic solutes.

Various additives that increase the stability and sterility of the compositions, including antimicrobial preservatives, antioxidants, chelating agents, and buffers, can be added. The action of microorganisms can be reduced by various antibacterial and antifungal agents, for example, parabens, chlorobutanol, phenol, and sorbic acid.

In some embodiments, the pharmaceutical composition comprises a pharmaceutically-acceptable excipient, a pharmaceutically-acceptable carrier, a diluent, adjuvant, stabilizer, emulsifier, preservative, colorant, buffer, or a combination thereof. In some embodiments, the pharmaceutical composition comprises a chemotherapeutic agent, an immunosuppressive agent, an immuno-stimulatory agent, an anti-pyretic agent, a cytotoxic agent, a nucleolytic compound, a radioactive isotope, a receptor, a pro-drug activating enzyme, an anti-inflammatory agent, an antibiotic, a protease inhibitor, a growth factor, an antibacterial agent, an antifungal agent, or a combination thereof. Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

The active amniotic fluid stem cells can be administered topically and can be formulated into a variety of topically-administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The initial administration can be via any route practical, such as by any route described herein using any formulation described herein. A therapeutic agent can be administered as soon as is practicable after the onset of a disease or condition is detected or suspected. A pharmaceutical composition disclosed herein can be used, for example, before, during, or after treatment of a subject with another pharmaceutical agent.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

In some embodiments, a pharmaceutically-acceptable amount of a composition of the disclosure is administered to a subject gradually over a period of time. In some embodiments, an amount of a composition of the disclosure can be administered to a subject gradually over a period of from about 0.1 h to about 24 h. In some embodiments, an amount of a composition of the disclosure can be administered to a subject over a period of about 0.1 h, about 0.2 h, about 0.3 h, about 0.4 h, about 0.5 h, about 0.6 h, about 0.7 h, about 0.8 h, about 0.9 h, about 1 h, about 1.5 h, about 2 h, about 2.5 h, about 3 h, about 3.5 h, about 4 h, about 4.5 h, about 5 h, about 5.5 h, about 6 h, about 6.5 h, about 7 h, about 7.5 h, about 8 h, about 8.5 h, about 9 h, about 9.5 h, about 10 h, about 10.5 h, about 11 h, about 11.5 h, about 12 h, about 12.5 h, about 13 h, about 13.5 h, about 14 h, about 14.5 h, about 15 h, about 15.5 h, about 16 h, about 16.5 h, about 17 h, about 17.5 h, about 18 h, about 18.5 h, about 19 h, about 19.5 h, about 20 h, about 20.5 h, about 21 h, about 21.5 h, about 22 h, about 22.5 h, about 23 h, about 23.5 h, or about 24 h. In some embodiments, a pharmaceutically-acceptable amount of a composition of the disclosure is administered gradually over a period of about 0.5 h. In some embodiments, a pharmaceutically-acceptable amount of a composition of the disclosure is administered gradually over a period of about 1 h. In some embodiments, a pharmaceutically-acceptable amount of a composition of the disclosure is administered gradually over a period of about 1.5 h.

Pharmaceutical compositions described herein can be administered 1, 2, 3, 4, 5, 6, 7, 8, 9, 10, 11, 12, 13, 14, 15, 16, 17, 18, 19, 20 or more times, and can be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6.7 days, or 1, 2, 3, 4, 5 weeks, or 1, 2, 3, 4, 5, 6, 7, 8, 9, 10, 11, or 12 months.

The disclosed amniotic fluid stem cells disclosed herein can also be banked and proliferated in culture as needed, as a convenient and readily available source for therapies in recipient subjects.

Compositions of the disclosure can be administered to a subject by any suitable route. Non-limiting examples of routes of administration include topical, transdermal, muscular, intramuscular, sub-muscular, inhaled, parenteral, intravenous, intra-lymphatic, intra-lesion, intra-tumoral, intra-articular, epidural, subcutaneous, mucosal, and sub-mucosal routes. A composition can be administered as a bolus. A composition can be administered by infusion.

Administering can be performed, for example, once as a single dose, or a plurality of times as a plurality of doses. In some embodiments, the administering can be performed over one or more extended periods of times (e.g., over a day, a week, a month, a year, or multiples thereof) either as a single dose or as a plurality of doses.

Compositions described herein can be in unit dosage forms suitable for administration of precise dosages. In unit dosage form, the formulation can be divided into or dispensed as unit doses containing appropriate quantities of cells. The unit dosage can be in the form of a package containing discrete quantities of the formulation. Non-limiting examples are and liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi dose containers with a preservative.

In practicing the methods of treatment or use provided herein, therapeutically-effective amounts of the amniotic fluid stem cells described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, and other factors. The amniotic fluid stem cells can be used singly or in combination with one or more therapeutic agents as components of mixtures.

In some embodiments, the administering is performed daily for a period of at least one week. In some embodiments, the administering is performed weekly for a period of at least one month. In some embodiments, the administering is performed monthly for a period of at least 2 months. In some embodiments, the administering is performed daily, weekly, or monthly for a period of at least one year. In some embodiments, the administering is performed at least once monthly. In some such embodiments, the administering is performed between 1 and 2 times per month. In some embodiments, the administering is performed at least once weekly.

A dose of amniotic fluid stem cells of the disclosure can be about: 1×10¹ cells, about 1×10² cells, about 1×10³ cells, about 1×10⁴ cells, about 1×10⁵ cells, about 1×10⁶ cells, about 1×10⁷ cells, about 1×10⁸ cells, about 1×10⁹ cells, about 1×10¹⁰ cells, about 1×10¹¹ cells, or about 1×10¹² cells. In some embodiments, a dose of amniotic fluid stem cells can be at least about: 1×10¹ cells, at least about 1×10² cells, at least about 1×10³ cells, at least about 1×10⁴ cells, at least about 1×10⁵ cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more. In some embodiments, a dose of amniotic fluid stem cells can be at most about: 1×10¹ cells, at most about 1×10² cells, at most about 1×10³ cells, at most about 1×10⁴ cells, at most about 1×10⁵ cells, at most about 1×10⁶ cells, at most about 1×10⁷ cells, at most about 1×10⁸ cells, at most about 1×10⁹ cells, at most about 1×10¹⁰ cells, at most about 1×10¹¹ cells, at most about 1×10¹² cells, or less.

In some cases, a dose of amniotic fluid stem cells is about: 1×10¹ to about 1×10² cells, about 1×10² to about 1×10³ cells, about 1×10³ to about 1×10⁴ cells, about 1×10⁴ to about 1×10⁵ cells, about 1×10⁵ to about 1×10⁶ cells, about 1×10⁶ to about 1×10⁷ cells, about 1×10⁷ to about 1×10⁸ cells, about 1×10⁸ to about 1×10⁹ cells, about 1×10⁹ to about 1×10¹⁰ cells/kg, about 1×10¹⁰ to about 1×10¹¹ cells/kg, or about 1×10¹¹ to about 1×10¹² cells.

A dose of amniotic fluid stem cells of the disclosure can vary based on the weight of a recipient subject. For example, a dose of amniotic fluid stem cells can be about: 1×10¹ cells/kg, about 1×10² cells/kg, about 1×10³ cells/kg, about 1×10⁴ cells/kg, about 1×10⁵ cells/kg, about 1×10⁶ cells/kg, about 1×10⁷ cells/kg, about 1×10⁸ cells/kg, about 1×10⁹ cells/kg, about 1×10¹⁰ cells/kg, about 1×10¹¹ cells/kg, or about 1×10¹² cells/kg. In some embodiments, a dose of amniotic fluid stem cells can be at least about: 1×10¹ cells/kg, at least about 1×10² cells/kg, at least about 1×10³ cells/kg, at least about 1×10⁴ cells/kg, at least about 1×10⁵ cells/kg, at least about 1×10⁶ cells/kg, at least about 1×10⁷ cells/kg, at least about 1×10⁸ cells/kg, at least about 1×10⁹ cells/kg, at least about 1×10¹⁰ cells/kg, at least about 1×10¹¹ cells/kg, at least about 1×10¹² cells/kg, or more. In some embodiments, a dose of amniotic fluid stem cells can be at most about: 1×10¹ cells/kg, at most about 1×10² cells/kg, at most about 1×10³ cells/kg, at most about 1×10⁴ cells/kg, at most about 1×10⁵ cells/kg, at most about 1×10⁶ cells/kg, at most about 1×10⁷ cells/kg, at most about 1×10⁸ cells/kg, at most about 1×10⁹ cells/kg, at most about 1×10¹⁰ cells/kg, at most about 1×10¹¹ cells/kg, at most about 1×10¹² cells/kg, or less.

In some cases, a dose of amniotic fluid stem cells is about: 1×10¹ to about 1×10² cells/kg; about 1×10² to about 1×10³ cells/kg; about 1×10³ to about 1×10⁴ cells/kg; about 1×10⁴ to about 1×10⁵ cells/kg; about 1×10⁵ to about 1×10⁶ cells/kg; about 1×10⁶ to about 1×10⁷ cells/kg; about 1×10⁷ to about 1×10⁸ cells/kg; about 1×10⁸ to about 1×10⁹ cells/kg; about 1×10⁹ to about 1×10¹⁰ cells/kg, about 1×10¹⁰ to about 1×10¹¹ cells/kg, or about 1×10¹¹ to about 1×10¹² cells/kg.

Amniotic fluid stem cells of the disclosure can be suspended in a volume suitable for administration. For example, the cells can be suspended in a volume of about: 0.1 ml, about 0.2 ml, about 0.3 ml, about 0.4 ml, about 0.5 ml, about 0.6 ml, about 0.7 ml, about 0.8 ml, about 0.9 ml, about 1 ml, about 2 ml, about 3 ml, about 4 ml, about 5 ml, about 6 ml, about 7 ml, about 8 ml, about 9 ml, about 10 ml, about 11 ml, about 12 ml, about 13 ml, about 14 ml, about 15 ml, about 16 ml, about 17 ml, about 18 ml, about 19 ml, about 20 ml, about 21 ml, about 22 ml, about 23 ml, about 24 ml, about 25 ml, about 26 ml, about 27 ml, about 28 ml, about 29 ml, about 30 ml, about 31 ml, about 32 ml, about 33 ml, about 34 ml, about 35 ml, about 36 ml, about 37 ml, about 38 ml, about 39 ml, about 40 ml, about 41 ml, about 42 ml, about 43 ml, about 44 ml, about 45 ml, about 46 ml, about 47 ml, about 48 ml, about 49 ml, about 50 ml, about 60 ml, about 70 ml, about 80 ml, about 90 ml, about 100 ml, about 200 ml, about 300 ml, about 400 ml, or about 500 ml. In some embodiments, amniotic fluid stem cells can be suspended in a volume of about 0.1 ml to about 1 ml; about 1 ml to about 10 ml; about 10 ml to about 50 ml; about 50 ml to about 100 ml; about 100 ml to about 200 ml; about 200 ml to about 300 ml; about 300 ml to about 400 ml; or about 400 ml to about 500 ml.

In some embodiments, amniotic fluid stem cells can be administered to a subject in a volume that varies depending upon the weight of the subject. For example, amniotic fluid stem cells of the disclosure can be administered in a volume of about 0.01 ml, about 0.1 ml, about 0.5 ml, about 1 ml, about 2 ml, about 3 ml, about 4 ml, about 5 ml, about 10 ml, about 20 ml, about 30 ml, about 40 ml or about 50 ml per kg recipient body weight, e.g., in a volume of about 0.01 to about 0.1, about 0.1 to about 1, about 1 to about 2; about 2 to about 3; about 3 to about 4; about 4 to about 5; about 1 to about 5; about 5 to about 10; about 10 to about 20; about 20 to about 30; about 30 to about 40; or about 40 to about 50 ml per kg recipient body weight.

Amniotic fluid stem cells of the disclosure can be used in the manufacture of engineered tissue and organs, including structures such as scaffolds, patches or plugs of tissues or matrix material, prosthetics, other implants, repair or dressing of wounds, hemostatic devices, devices for use in tissue repair and support such as sutures, surgical and orthopedic screws, and surgical plates, orthopedic plates, natural coatings or components for synthetic implants, cosmetic implants and supports, repair or structural support for organs or tissues, substance delivery, bioengineering platforms, platforms for testing the effect of substances upon cells, cell culture, and fetuses.

In some embodiments, the shape of a composition helps send signals to the cells to grow and function in a specific way. Other substances, for example differentiation inducers, can be added to the compositions to promote specific types of cell growth. The ability to use the amniotic fluid stem cells of the disclosure to bioengineer tissue or organs facilitates a wide variety of bioengineered tissue replacement applications. Non-limiting examples of bioengineered components include bone, dental structures, joints, cartilage, skeletal muscle, smooth muscle, cardiac muscle, tendons, knees, menisci, ligaments, blood vessels, stents, heart valves, corneas, ear drums, nerve guides, tissue or organ patches or sealants, a filler for missing tissues, sheets for cosmetic repairs, skin, soft tissue structures of the throat such as trachea, epiglottis, and vocal cords, other cartilaginous structures such as nasal cartilage, tarsal plates, tracheal rings, thyroid cartilage, arytenoid cartilage, connective tissue, vascular grafts and components thereof, sheets for topical applications, and components that can contribute to repair or replacement of organs such as liver, kidney, lung and pancreas.

In some embodiments, amniotic fluid stem cells of the disclosure are combined or co-administered with drugs to improve transplantation. For example, antibiotics, anti-inflammatory agents, local anesthetics, immunosuppressive agents, or combinations thereof, can be added to the matrix of a bioengineered organ to speed the healing process and reduce pain and discomfort. In some embodiments, the disclosed scaffolds can further comprise one or more biological agents. The biological agents can provide the biodegradable scaffolds with mechanical strength and control over their mechanical and degradation properties. Non-limiting examples of biological agents include fibronectin, collagen and gelatin, and synthetic polymers, such as a flexible nanofiber, polyethylene glycol or a polylactic glycolide.

Amniotic fluid stem cells of the disclosure can be applied in a scaffold of various materials appropriate to the site or organ, or tissue being repaired, replaced, or regenerated, for example, a collagen or hydrogel matrix. A scaffold can provide a structural support for stem cell adhesion, proliferation and differentiation, and can serve as a microenvironment for guiding stem cell differentiation, regeneration, and structure. Non-limiting examples of natural scaffolds used in tissue engineering include collagen, silk fibroin, alginate, chitosan, keratin, and decellularized tissues such as de-epithelialized human amniotic membrane. Synthetic scaffolds can be made of biodegradable polymers. Biocompatible scaffolds can be precursors to implantable devices, which can have the ability to perform an intended function without eliciting undesirable effects in the stem cells or inducing any undesirable local or systemic responses in the eventual host.

A hydrogel can be a water-swellable polymeric matrix that can absorb a substantial amount of water to form elastic gels. The matrix can be a three-dimensional network of macromolecules held together by covalent or non-covalent crosslinks. Upon placement in an aqueous environment, dry hydrogels can swell to the extent allowed by the degree of cross-linking. A hydrogel composition can include one or more non-hydrogel components or compositions, e.g., hydrocolloids, which contain a hydrophilic component (which can contain or be a hydrogel) distributed in a hydrophobic phase.

Conditions

Disclosed herein are compositions and methods that can be used for treating a condition in a subject in need thereof. For example, compositions and methods disclosed herein can be used for treating, reducing, correcting, managing, controlling, or ameliorating a condition characterized by a damaged tissue, a dysfunctional organ, or a combination thereof. In some embodiments, compositions and methods disclosed herein can be used for treating, reducing, correcting, managing, controlling, or ameliorating congenital malformations of a fetus.

In some embodiments, compositions of the disclosure are suitable for reducing, slowing, or stopping the development of a condition, for example, in a subject who is at risk of developing or has a particular condition. The subject can be a mammal. The subject can be a human. The subject can be a non-human animal. The subject can be any age. For example, the subject can be a fetus, a neonate, an infant, a child, an adolescent, a young adult, an adult, or an elderly subject. In some embodiments, the subject is a fetus of a gestational age of at least about 1, at least about 2, at least about 3, at least about 4, at least about 5, at least about 6, at least about 7, at least about 8, or at least about 9 months. In some embodiments, the subject is a fetus of a gestational age of at most about 1, at most about 2, at most about 3, at most about 4, at most about 5, at most about 6, at most about 7, at most about 8, or at most about 9 months. In some embodiments, the subject is at least about 1, at least about 2, at least about 3, at least about 4, at least about 5, at least about 6, at least about 7, at least about 8, at least about 9, at least about 10, at least about 12, or at least about 18 months old. In some embodiments, the subject is at most about 1, at most about 2, at most about 3, at most about 4, at most about 5, at most about 6, at most about 7, at most about 8, at most about 9, at most about 10, at most about 12, or at most about 18 months old. In some embodiments, the subject is at least about 1, at least about 2, at least about 3, at least about 4, at least about 5, at least about 6, at least about 7, at least about 8, at least about 9, at least about 10, at least about 12, at least about 14, at least about 16, at least about 18, at least about 20, at least about 21, at least about 23, at least about 25, at least about 30, at least about 35, at least about 40, at least about 45, at least about 50, at least about 55, at least about 60, at least about 65, at least about 70, at least about 75, or at least about 80 years old. In some embodiments, the subject is at most about 1, at most about 2, at most about 3, at most about 4, at most about 5, at most about 6, at most about 7, at most about 8, at most about 9, at most about 10, at most about 12, at most about 14, at most about 16, at most about 18, at most about 20, at most about 21, at most about 23, at most about 25, at most about 30, at most about 35, at most about 40, at most about 45, at most about 50, at most about 55, at most about 60, at most about 65, at most about 70, at most about 75, at most about or 80 years old.

Amniotic fluid stem cells of the disclosure that are administered to a subject can be from a donor. For example, amniotic fluid stem cells can be harvested from a donor, processed using methods disclosed herein to generate a suitable population of amniotic fluid stem cells, and administered to the subject. The amniotic fluid stem cells can be autologous to the subject (e.g., the subject is the donor). The amniotic fluid stem cells can be allogenic to the subject (e.g., from a non-genetically identical donor). The amniotic fluid stem cells can be HLA matched to the subject (e.g., matched for at least one HLA allele, such as an HLA-A allele, an HLA-B allele, an HLA-C allele, an HLA-DP allele, an HLA-DQ allele, an HLA-DR allele, or any combination thereof). In some embodiments, the amniotic fluid stem cells are fully HLA matched to the subject. In some embodiments, the amniotic fluid stem cells are haploidentical to the subject. In some embodiments, the amniotic fluid stem cells are from a relative (e.g., a sibling donor). In some embodiments, the amniotic fluid stem cells are partly HLA mismatched to the subject (e.g., mismatched for at least one HLA allele). In some embodiments, the amniotic fluid stem cells are fully HLA mismatched to the subject.

In some embodiments, compositions and methods are provided that can be used for treating Alzheimer's disease. Alzheimer's disease can be an irreversible, progressive brain disorder that slowly destroys memory and thinking skills, and eventually the ability to carry out the simplest tasks. AD can be associated with the formation of β amyloid plaques and neurofibrillary tangles of the tau protein in the brain and the loss of connections between neurons in the brain. The damage can initially take place in the hippocampus, and as more neurons die, additional parts of the brain can be affected and begin to shrink. Memory problems can be one of the first signs of cognitive impairment related to Alzheimer's disease. As AD progresses, memory loss confusion and inability to recognize familiar faces can worsen. Ultimately, plaques and tangles spread throughout the brain, and brain tissue shrinks significantly. Causes of AD can include a combination of genetic, environmental, and lifestyle factors.

In some embodiments, compositions and methods are provided that can be used for treating Amyotrophic Lateral Sclerosis (ALS). ALS can be a progressive neurodegenerative disease that affects motor neurons in the brain and the spinal cord, with consequent muscle degeneration and atrophy.

In some embodiments, compositions and methods are provided that can be used for treating cancer. Cancer can be a condition characterized by unregulated cell growth. Non-limiting examples of cancer include acute leukemia, astrocytomas, basal cell carcinoma, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, Wilms' tumors, sarcoma of soft tissue, schwannoma, skin cancer, spinal axis tumors, squamous cell carcinomas, stomach cancer, synovial sarcoma, testicular cancer, uterine cancer, or tumors and their metastases, including refractory versions of any of the above cancers, and any combination thereof.

In some embodiments, compositions and methods are provided that can be used for treating Huntington's Disease (HD). HD can be a fatal genetic disorder that causes the progressive breakdown of nerve cells in the brain and the deterioration of a person's physical and mental abilities. Every child of a parent with HD can have a 50/50 chance of carrying the faulty gene. Symptoms can appear between the ages of 30 to 50, and worsen over a 10 to 25 year period. Symptoms can include personality changes, mood swings, depression, forgetfulness, impaired judgment, unsteady gait, involuntary movements, slurred speech, difficulty in swallowing, and significant weight loss.

In some embodiments, compositions and methods are provided that can be used for treating Multiple Sclerosis (MS). Multiple Sclerosis can be a progressive neurodegenerative disorder that involves an immune-mediated process in which the body's immune system is directed against myelin, a protective coating of nerve fibers in the CNS, the nerve fibers and the cells that produce myelin. The damage can produce a variety of neurological symptoms.

In some embodiments, compositions and methods are provided that can be used for treating Multiple System Atrophy (MSA). Multiple System Atrophy can be a rare, degenerative neurologic condition that affects cells that produce dopamine, a neurotransmitter that controls motor commands. MSA can affect both neurons and glial cells.

In some embodiments, compositions and methods are provided that can be used for treating neonatal encephalopathy. Neonatal encephalopathy can be a neonatal ischemic brain injury, which can cause permanent motor-deficit cerebral palsy and potentially death.

In some embodiments, compositions and methods are provided that can be used for treating a neurodegenerative disease. A neurodegenerative disease can be an acute or chronic condition, disorder, or disease of the central or peripheral nervous system. A neurodegenerative condition can be age-related, can result from injury or trauma, and/or can be related to a specific disease or disorder. Non-limiting examples of acute neurodegenerative conditions include conditions associated with neuronal cell death or compromise, including cerebrovascular insufficiency, focal or diffuse brain trauma, diffuse brain damage, spinal cord injury or peripheral nerve trauma. Non-limiting examples of acute neurodegenerative disorders include cerebral ischemia, infarction, embolic occlusion, thrombotic occlusion, reperfusion following acute ischemia, perinatal hypoxic-ischemic injury, cardiac arrest, intracranial hemorrhage, intracranial and intravertebral lesions, whiplash, and shaken infant syndrome. Non-limiting examples chronic neurodegenerative conditions include Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (for example, Creutzfeldt-Jakob, Gerstmann-Straussler-Scheinker disease, Kuru and fatal familial insomnia), demyelination diseases, and disorders including multiple sclerosis and hereditary diseases such as leukodystrophies.

In some embodiments, compositions and methods are provided that can be used for treating Parkinson's Disease. Parkinson's disease can be a brain disorder that leads to shaking, stiffness, difficulty with walking, balance, and coordination, mental and behavioral changes, sleep problems, depression, memory difficulties, and fatigue. Parkinson's symptoms can begin gradually and get worse over time. PD can be triggered when neurons in the brain die, with consequent reductions in the production of dopamine and norepinephrine. The lack of dopamine can cause the movement problems associated with PD, and the loss of norepinephrine can lead to fatigue, irregular blood pressure, decreased movement of food through the digestive tract, and sudden drop in blood pressure when a person stands up from a sitting or lying-down position. Non-limiting examples symptoms of PD include tremor in hands, arms, legs, jaw, or head; stiffness of the limbs and trunk; slowness of movement; impaired balance and coordination; depression; difficulty swallowing, chewing, and speaking; urinary incontinence or constipation, skin problems; and sleep disruptions. The main therapy for Parkinson's can be levodopa to produce dopamine, in combination with carbidopa to prevent or reduce some of the side effects of levodopa. Once this therapy is no longer effective, subjects with PD can be treated with dopamine agonists, MAO-B inhibitors, COMT inhibitors, Amantadine, and/or anticholinergic drugs to slow progression of the disease.

In some embodiments, compositions and methods are provided that can be used for treating Periventricular Leukomalacia. Periventricular Leukomalacia can be a type of brain injury that affects premature infants. The condition can involve death of small areas of brain tissue around the ventricles.

In some embodiments, compositions and methods are provided that can be used for treating a congenital malformation. The congenital malformation can be a genetic disorder, a tumor, an arrest of an organ development, or a result from exposure to a toxin, smoke, alcohol or fetal injury during pregnancy. Transplantation can be in utero or after birth. In some embodiments, a congenital malformation is a bone or cartilage defect, and amniotic fluid stem cells of the disclosure differentiate into osteoblasts or osteocytes (e.g., differentiate in vivo after administration, and/or differentiate partly in an osteogenic medium before administration to the subject).

In some embodiments, the congenital malformation is a heart tissue malformation, and the amniotic fluid stem cells of the disclosure differentiate into myofibroblast and endothelial cells (e.g., differentiate in vivo after administration, and/or differentiate partly in a myogenic medium before administration to the subject).

In some embodiments, the congenital malformation is a heart defect, and amniotic fluid stem cells of the disclosure differentiate into myocardial cells (e.g., differentiate in vivo after administration, and/or differentiate partly in a myogenic medium before administration to the subject).

In some embodiments, the congenital malformation is a kidney malformation, and amniotic fluid stem cells of the disclosure differentiate into renal cells (e.g., differentiate in vivo after administration, and/or differentiate partly in a nephrogenic medium before administration to the subject).

In some embodiments, the congenital malformation is a lung defect, and amniotic fluid stem cells of the disclosure differentiate into epithelial and mesenchymal cells (e.g., differentiate in vivo after administration, and/or differentiate partly in an alveolargenic medium before administration to the subject).

In some embodiments, the congenital malformation is periventricular leukomalacia, and amniotic fluid stem cells of the disclosure differentiate into angiogenic cells (e.g., differentiate in vivo after administration, and/or differentiate partly in an angiogenic medium before administration to the subject).

In some embodiments, the congenital malformation is neonatal encephalopathy, and amniotic fluid stem cells of the disclosure into angiogenic cells (e.g., differentiate in vivo after administration, and/or differentiate partly in an angiogenic medium before administration to the subject).

In some embodiments, the congenital malformation is traumatic brain injury, and amniotic fluid stem cells of the disclosure differentiate into neurons (e.g., differentiate in vivo after administration, and/or differentiate partly in a neurogenic medium before administration to the subject).

Amniotic fluid stem cells of the disclosure can treat diseases associated traumatic injuries and dysfunctional tissues and organs. The cells are capable of migrating to the site of an injury upon administration to a subject, and regenerating damaged tissues and organs. In some embodiments, provided herein are methods of repairing a tissue or replacing an organ in a subject in need thereof, wherein the methods comprise administering to the subject a pharmaceutical composition that comprises amniotic fluid stem cells of the disclosure to regenerate the tissue or organ, thereby repairing the tissue or replacing the organ in the subject.

In some embodiments, the tissue or organ is part of the subject's respiratory tract, gastrointestinal tract, salivary glands, cardiovascular system, liver, pancreas, bone marrow, joints, bones, cartilage, knee, skeleton, central nervous system or skin. Administration of the pharmaceutical composition can be topical, transdermal, mucosal, sub-mucosal, muscular, sub-muscular, by inhalation, parenteral or intravenous administration.

In some embodiments, provided herein are methods of managing or treating neonatal encephalopathy, traumatic brain injury or ischemia in a subject in need thereof, wherein the methods comprise administering to the subject a pharmaceutical composition that comprises a therapeutically-effective amount of amniotic fluid stem cells of the disclosure, thereby managing or treating neonatal encephalopathy, traumatic brain injury or ischemia in the subject.

In some embodiments, provided herein are methods of treating, controlling or managing diabetes in a subject with a damaged pancreas, wherein the methods comprise regenerating pancreatic islets by administering to the subject a therapeutically-effective amount of a pharmaceutical composition that comprises amniotic fluid stem cells of the disclosure, thereby treating, controlling, or managing diabetes in the subject.

In some embodiments, provided herein are methods of treating, controlling or managing a cardiovascular disease in a subject in need thereof, wherein the methods comprise regenerating cardiac tissue and promoting vascularization by injecting into the subject's cardiac tissue a therapeutically-effective amount of a pharmaceutical composition that comprises the amniotic fluid stem cells of the disclosure, thereby treating, controlling, or managing the cardiovascular disease.

In some embodiments, provided herein are methods of treating, controlling, or managing a progressive neurodegenerative disease in a subject in need thereof, wherein the methods comprise regenerating neurons by injecting into the subject's brain a therapeutically-effective amount of a pharmaceutical composition that comprises amniotic fluid stem cells of the disclosure, thereby treating, controlling, or managing the progressive neurodegenerative disease. Non-limiting examples of progressive neurodegenerative diseases include Alzheimer's disease, Huntington's disease, amyotrophic lateral sclerosis, multiple sclerosis, multiple system atrophy, and Parkinson's disease.

In some embodiments, provided herein are methods of treating, controlling or managing muscular dystrophy in a subject in need thereof, wherein the methods comprise regenerating myogenic cells by administering to the subject a therapeutically-effective amount of a pharmaceutical composition that comprises amniotic fluid stem cells of the disclosure, thereby treating, controlling, or managing muscular dystrophy.

In some embodiments, provided herein are methods of managing, controlling or treating a peripheral nerve or muscle injury in a subject in need thereof, wherein the methods comprise parenterally administering to the subject a therapeutically-effective amount of a pharmaceutical composition that comprises amniotic fluid stem cells of the disclosure to regenerate the peripheral nerve or muscle, thereby managing, controlling, or treating a peripheral nerve or muscle injury in the subject.

In some embodiments, provided herein are methods of regenerating skin, repairing a burn, or healing a wound in a subject in need thereof, wherein the methods comprise topically or parenterally administering to the subject a therapeutically-effective amount of a pharmaceutical composition that comprises amniotic fluid stem cells of the disclosure, thereby regenerating the skin, repairing the burn, or healing the wound in the subject. The wound can be an ulcer. The wound can be a foot ulcer. The wound can be a diabetic ulcer.

In some embodiments, provided herein are methods of controlling, managing, or treating arthritis in a subject in need thereof, wherein the methods comprise administering to the subject a therapeutically-effective amount of a pharmaceutical composition that comprises the disclosed amniotic fluid stem cells of the disclosure, thereby controlling, managing, or treating arthritis.

In some embodiments, the disclosed methods can further comprise administering to the subject prior to, during or after transplantation a therapeutically-effective amount of one or more of a chemotherapeutic agent, an immunosuppressive agent, an immune-stimulatory agent, an anti-pyretic agent, a cytotoxic agent, a nucleolytic compound, a radioactive isotope, a receptor, a pro-drug activating enzyme, an anti-inflammatory agent, an antibiotic, a protease inhibitor, a growth factor, an osteo-inductive factor, an antibacterial agent, and an antifungal agent.

Bioassays

Disclosed herein, in some embodiments, are bioassays that utilize amniotic fluid stem cells of the disclosure. A bioassay can be an analytical method to determine safety, concentration, or potency of a substance by the effect of the substance on living cells or tissues. A bioassay can involve the application of a test agent, such as a drug, to a population of cells comprising amniotic fluid stem cells. Bioassays can be used to determine a particular effect of the test agent on the amniotic fluid stem cells, such as a change in cell proliferation or differentiation, growth, survival. In some embodiments, a bioassay can be used to determine the potential of a test agent for teratogenicity.

The use of drugs, nutritional agents, and potentially teratogenic substances by pregnant women can have major effects on the developing fetus. In some embodiments, a bioassay disclosed herein can be utilized to assess the effect of a test agent on fetal tissues and/or on human fetal growth and development. For example, a bioassay disclosed herein can be utilized to evaluate the effect of a drug, nutritional agent, or potentially teratogenic substance on fetal tissues and/or on human fetal growth and development.

A bioassay disclosed herein can comprise determining an effect of an agent on a population of amniotic fluid stem cells. A bioassay of the disclosure can comprise, for example, contacting a population of cells comprising amniotic fluid stem cells with a test agent, and determining the effect of the test agent on the amniotic fluid stem cells. Determining the effect of the agent on the clonal population of amniotic fluid stem cells can comprise determining any combination of parameters disclosed herein. For example, determining the effect of the agent on the clonal population of amniotic fluid stem cells can comprise measuring an amount of a reporter gene that is expressed in response to the contacting. Determining the effect of the agent on the clonal population of amniotic fluid stem cells can comprise measuring expression of a marker that is associated with differentiation, for example, ectodermal differentiation, mesodermal differentiation, endodermal differentiation, or a combination thereof. Determining the effect of the agent on the clonal population of amniotic fluid stem cells can comprise measuring expression of a marker that is associated with differentiation to a particular cell type lineage, for example, neuronal differentiation, chondrocyte differentiation, osteocyte differentiation, or a combination thereof. Determining the effect of the agent on the clonal population of amniotic fluid stem cells can comprise measuring survival of a population of amniotic fluid stem cells or a population of cells differentiation from amniotic fluid stem cells into cells of a certain germ layer or cell type lineage.

Bioassays can comprise any set of controls and conditions required to determine the effect of the test agent. For example, amniotic fluid stem cells of the disclosure can be contacted with the test agent for different amounts of time to evaluate how the duration of exposure affects the readout. Alternatively or additionally, amniotic fluid stem cells of the disclosure can be contacted with the test agent at different concentrations, for example, to determine a dose-response curve, an amount that exhibits toxicity, an amount that exhibits a therapeutic effect, an amount that promotes development, an amount that promotes differentiation, an amount that impairs development, an amount that impairs differentiation, or a combination thereof.

A bioassay disclosed herein can comprise use of any appropriate experimental assays, for example, assays comprising fluorescence microscopy, flow cytometry, monoclonal antibody binding, differential gene expression analysis, genomic analysis, transcriptomic analysis, metagenomic analysis, epigenetic analysis, enzymatic colorimetric assays, or any combination thereof.

A bioassay disclosed herein can comprise a reporter gene. For example, a population of amniotic fluid stem cells of the disclosure can comprise one or more dose-responsive reporter genes driven by an inducible promoter. A bioassay disclosed herein can comprise quantifying gene expression. A bioassay disclosed herein can comprise quantifying a level of expression of a marker or determining a proportion of cells that express a marker, e.g., by microscopy or cytometry. A bioassay disclosed herein can comprise morphologic evaluation of amniotic fluid stem cells. A bioassay disclosed herein can comprise the use of one or more spheroids comprising amniotic fluid stem cells as disclosed herein.

A test agent can be a chemical substance or compound that induces a desired pharmacological or physiological effect, and can include therapeutically effective, prophylactically effective, or systematically effective agents. A test agent can also encompass pharmaceutically acceptable, pharmacologically active derivatives and analogues of those active test agents disclosed herein, for example, salts, esters, amides, prodrugs, active metabolites, and inclusion complexes. Non-limiting examples of test agents suitable for incorporation into pharmaceutical compositions include adrenergic agents; adrenocortical steroids; adrenocortical suppressants; alcohol deterrents; aldosterone antagonists; amino acids; ammonia detoxicants; anabolic agents; analeptic agents; analgesic agents; androgenic agents; anesthetic agents; anorectic compounds; anorexic agents; antagonists; anterior pituitary activators and anterior pituitary suppressants; anti-acne agents; anti-adrenergic agents; anti-allergic agents; anti-amebic agents; anti-androgen agents; anti-anemic agents; anti-anginal agents; anti-anxiety agents; anti-arthritic agents; anti-asthmatic agents and other respiratory drugs; anti-atherosclerotic agents; anti-bacterial agents; anti-cancer agents, including antineoplastic drugs, and anti-cancer supplementary potentiating agents; anticholinergics; anticholelithogenic agents; anti-coagulants; anti-coccidal agents; anti-convulsants; anti-depressants; anti-diabetic agents; anti-diarrheals; anti-diuretics; antidotes; anti-dyskinetics agents; anti-emetic agents; anti-epileptic agents; anti-estrogen agents; anti-fibrinolytic agents; anti-fungal agents; anti-glaucoma agents; antihelminthics; anti-hemophilic agents; anti-hemophilic Factor; anti-hemorrhagic agents; antihistamines; anti-hyperlipidemic agents; anti-hyperlipoproteinemic agents; antihypertensive agents; anti-hypotensives; anti-infective agents such as antibiotics and antiviral agents; anti-inflammatory agents, both steroidal and non-steroidal; anti-keratinizing agents; anti-malarial agents; antimicrobial agents; anti-migraine agents; anti-mitotic agents; anti-mycotic agents; antinauseants; antineoplastic agents; anti-neutropenic agents; anti-obsessional agents; anti-parasitic agents; antiparkinsonism drugs; anti-pneumocystic agents; anti-proliferative agents; anti-prostatic hypertrophy drugs; anti-protozoal agents; antipruritics; anti-psoriatic agents; antipsychotics; antipyretics; antispasmodics; anti-rheumatic agents; anti-schistosomal agents; anti-seborrheic agents; anti-spasmodic agents; anti-tartar and anti-calculus agents; anti-thromb otic agents; anti-tubercular agents; antitussive agents; anti-ulcerative agents; anti-urolithic agents; antiviral agents; GERD medications, anxiolytics; appetite suppressants; attention deficit disorder (ADD) and attention deficit hyperactivity disorder (ADHD) drugs; bacteriostatic and bactericidal agents; benign prostatic hyperplasia therapy agents; blood glucose regulators; bone resorption inhibitors; bronchodilators; carbonic anhydrase inhibitors; cardiovascular preparations including anti-anginal agents, anti-arrhythmic agents, beta-blockers, calcium channel blockers, cardiac depressants, cardiovascular agents, cardioprotectants, and cardiotonic agents; central nervous system (CNS) agents; central nervous system stimulants; choleretic agents; cholinergic agents; cholinergic agonists; cholinesterase deactivators; coccidiostat agents; cognition adjuvants and cognition enhancers; cough and cold preparations, including decongestants; depressants; diagnostic aids; diuretics; dopaminergic agents; ectoparasiticides; emetic agents; enzymes which inhibit the formation of plaque, calculus or dental caries; enzyme inhibitors; estrogens; fibrinolytic agents; fluoride anticavity/antidecay agents; free oxygen radical scavengers; gastrointestinal motility agents; genetic materials; glucocorticoids; gonad-stimulating principles; hair growth stimulants; hemostatic agents; herbal remedies; histamine H2 receptor antagonists; hormones; hormonolytics; hypnotics; hypocholesterolemic agents; hypoglycemic agents; hypolipidemic agents; hypotensive agents; HMGCoA reductase inhibitors; immunizing agents; immunomodulators; immunoregulators; immunostimulants; immunosuppressants; impotence therapy adjuncts; inhibitors; keratolytic agents; leukotriene inhibitors; LHRH agonists; liver disorder treatments; luteolysin agents; memory adjuvants; mental performance enhancers; metal chelators such as ethylenediaminetetraacetic acid, tetrasodium salt; mitotic inhibitors; mood regulators; mucolytics; mucosal protective agents; muscle relaxants; mydriatic agents; narcotic antagonists; nasal decongestants; neuroleptic agents; neuromuscular blocking agents; neuroprotective agents; nicotine; NMDA antagonists; non-hormonal sterol derivatives; nutritional agents, such as vitamins, essential amino acids and fatty acids; ophthalmic drugs such as antiglaucoma agents; oxytocic agents; pain relieving agents; parasympatholytics; peptide drugs; plasminogen activators; platelet activating factor antagonists; platelet aggregation inhibitors; post-stroke and post-head trauma treatments; potentiators; progestins; prostaglandins; prostate growth inhibitors; proteolytic enzymes as wound cleansing agents; prothyrotropin agents; psychostimulants; psychotropic agents; radioactive agents; regulators; relaxants; repartitioning agents; scabicides; sclerosing agents; sedatives; sedative-hypnotic agents; selective adenosine A1 antagonists; serotonin antagonists; serotonin inhibitors; serotonin receptor antagonists; steroids, including progestogens, estrogens, corticosteroids, androgens and anabolic agents; smoking cessation agents; stimulants; suppressants; sympathomimetics; synergists; thyroid hormones; thyroid inhibitors; thyromimetic agents; tranquilizers; tooth desensitizing agents; tooth whitening agents such as peroxides, metal chlorites, perborates, percarbonates, peroxyacids, and combinations thereof; unstable angina agents; uricosuric agents; vasoconstrictors; vasodilators including general coronary, peripheral and cerebral; vulnerary agents; wound healing agents; xanthine oxidase inhibitors; drugs of abuse, alcohols, and radiation.

### EMBODIMENTS

Embodiment 1. A composition comprising a population of amniotic fluid stem cells, wherein at least 65% of cells in the population of amniotic fluid stem cells are CD90 positive, wherein the population of amniotic fluid stem cells is in a unit dosage form, wherein the population comprises at least 50 cells.

Embodiment 2. A pharmaceutical composition comprising a population of amniotic fluid stem cells and a pharmaceutically-acceptable excipient, wherein at least 65% of cells in the population of amniotic fluid stem cells are CD90 positive.

Embodiment 3. A composition comprising a population of amniotic fluid stem cells, wherein at least 90% of cells in the population of amniotic fluid stem cells are CD90 positive, wherein at least 90% of cells in the population of amniotic fluid stem cells remain CD90 positive after two weeks in culture.

Embodiment 4. A composition comprising a clonal population of amniotic fluid stem cells, wherein the clonal population of amniotic fluid stem cells has been prepared by a process comprising: (a) isolating a single CD90 positive amniotic fluid stem cell from a plurality of amniotic fluid stem cells based on expression of CD90; and (b) expanding the single CD90 positive amniotic fluid stem cell to generate the clonal population of amniotic fluid stem cells.

Embodiment 5. A composition comprising a population of amniotic fluid stem cells, wherein the population of amniotic fluid stem cells has been prepared by a process comprising: (a) sorting a population of cells comprising amniotic fluid stem cells to obtain an enriched population of amniotic fluid stem cells comprising at least 90% CD90 positive cells; (b) expanding the enriched population of amniotic fluid stem cells in culture to obtain an expanded population of amniotic fluid stem cells; (c) sorting the expanded population of amniotic fluid stem cells to obtain a second enriched population of amniotic fluid stem cells comprising at least 90% CD90 positive cells; and (d) expanding the second enriched population of amniotic fluid stem cells in culture to obtain a second expanded population of amniotic fluid stem cells.

Embodiment 6. The composition of any one of the preceding embodiments, wherein at least 3% of cells in the population of amniotic fluid stem cells are TRA-1-60 positive.

Embodiment 7. The composition of any one of the preceding embodiments, wherein 3-30% of cells in the population of amniotic fluid stem cells are TRA-1-60 positive.

Embodiment 8. The composition of any one of the preceding embodiments, wherein at least 10% of cells in the population of amniotic fluid stem cells are SSEA4 positive.

Embodiment 9. The composition of any one of the preceding embodiments, wherein 10-50% of cells in the population of amniotic fluid stem cells are SSEA4 positive.

Embodiment 10. The composition of any one of the preceding embodiments, wherein at least 95% of cells in the population of amniotic fluid stem cells are CD90 positive.

Embodiment 11. The composition of any one of the preceding embodiments, wherein at least 5% of cells in the population of amniotic fluid stem cells are positive for CD117, CD15, CD44, CD29, CD9, CD73, CD105, CD133, or a combination thereof.

Embodiment 12. The composition of any one of the preceding embodiments, wherein at least 95% of cells in the population of amniotic fluid stem cells are CD29 positive.

Embodiment 13. The composition of any one of the preceding embodiments, wherein at least 90% of cells in the population of amniotic fluid stem cells are CD73 positive.

Embodiment 14. The composition of any one of the preceding embodiments, wherein at least 95% of cells in the population of amniotic fluid stem cells are CD29 positive.

Embodiment 15. The composition of any one of the preceding embodiments, wherein at least 10% of cells in the population of amniotic fluid stem cells are CD105 positive.

Embodiment 16. The composition of any one of the preceding embodiments, wherein at least 5% of cells in the population of amniotic fluid stem cells express Oct-4, Sox-2, Nanog, Rex-1, Prom1, or a combination thereof.

Embodiment 17. The composition of any one of the preceding embodiments, wherein at most 10% of cells in the population of amniotic fluid stem cells are CD117 positive.

Embodiment 18. The composition of any one of the preceding embodiments, wherein at most 10% of cells in the population of amniotic fluid stem cells are SSEA3 positive.

Embodiment 19. The composition of any one of the preceding embodiments, wherein at least 65% of cells in the population of amniotic fluid stem cells remain CD90 positive after two weeks in an in vitro cell culture.

Embodiment 20. The composition of any one of the preceding embodiments, wherein at least 3% of cells in the population of amniotic fluid stem cells remain TRA-1-60 positive after two weeks in in an in vitro cell culture.

Embodiment 21. The composition of any one of the preceding embodiments, wherein at least 10% of cells in the population of amniotic fluid stem cells remain SSEA4 positive after two weeks in in an in vitro cell culture.

Embodiment 22. The composition of any one of the preceding embodiments, wherein at least 50% of cells in the composition are the amniotic fluid stem cells.

Embodiment 23. The composition of any one of the preceding embodiments, wherein at least 90% of cells in the composition are the amniotic fluid stem cells.

Embodiment 24. The composition of any one of the preceding embodiments, wherein at least 95% of cells in the composition are the amniotic fluid stem cells.

Embodiment 25. The composition of any one of the preceding embodiments, wherein at least 99% of cells in the composition are the amniotic fluid stem cells.

Embodiment 26. The composition of any one of the preceding embodiments, wherein the population of amniotic fluid stem cells comprises at least 1×10\hat{ }5 cells.

Embodiment 27. The composition of any one of the preceding embodiments, wherein the composition comprises at least 1×10\hat{ }5 cells per mL.

Embodiment 28. The composition of any one of the preceding embodiments, wherein the amniotic fluid stem cells are progeny of amniotic fluid stem cells harvested from a pregnant human during the pregnant human's second trimester of pregnancy and cultured in vitro.

Embodiment 29. The composition of any one of the preceding embodiments, wherein the amniotic fluid stem cells are progeny of amniotic fluid stem cells harvested from a pregnant human during the pregnant human's third trimester of pregnancy and cultured in vitro.

Embodiment 30. The composition of any one of the preceding embodiments, wherein the amniotic fluid stem cells are progeny of amniotic fluid stem cells harvested from a pregnant human up to one week prior to a caesarian section and cultured in vitro.

Embodiment 31. The composition of any one of the preceding embodiments, wherein the population of amniotic fluid stem cells comprises cells that have been cultured in vitro for at least 7 days.

Embodiment 32. The composition of any one of the preceding embodiments, wherein the population of amniotic fluid stem cells comprises cells that have divided at least 10 times in vitro.

Embodiment 33. The composition of any one of the preceding embodiments, wherein at least 80% of the cells in the population are viable.

Embodiment 34. The composition of any one of the preceding embodiments, wherein the population of amniotic fluid stem cells comprises cells that differentiate into neurons as determined by an increase in nestin, beta tubulin III, and glial fibrillary acidic protein expression after incubation in neural induction medium.

Embodiment 35. The composition of any one of the preceding embodiments, wherein the population of amniotic fluid stem cells comprises cells that differentiate into chondrocytes as determined by an increase in aggrecan after incubation in chondrocyte induction medium.

Embodiment 36. The composition of any one of the preceding embodiments, wherein the population of amniotic fluid stem cells comprises cells that differentiate into osteocytes as determined by measuring alkaline phosphatase activity after incubation in osteocyte induction medium.

Embodiment 37. The composition of any one of the preceding embodiments, wherein the population of amniotic fluid stem cells comprises cells that differentiate into endodermal lineages as determined by an increase in SOX17 and CXCR4 expression after incubation in endodermal induction medium.

Embodiment 38. The composition of any one of the preceding embodiments, wherein the population of amniotic fluid stem cells comprises cells that differentiate into mesodermal lineages after incubation in mesodermal induction medium.

Embodiment 39. The composition of any one of the preceding embodiments, wherein the population of amniotic fluid stem cells comprises cells that differentiate into ectodermal lineages after incubation in ectodermal induction medium.

Embodiment 40. The composition of any one of the preceding embodiments, wherein the population of amniotic fluid stem cells comprises cells that differentiate into endodermal lineages, cells that differentiate into mesodermal lineages, and cells that differentiate into ectodermal lineages upon exposure to appropriate induction signals.

Embodiment 41. The composition of any one of the preceding embodiments, wherein after two weeks in culture in vitro, the population of amniotic fluid stem cells comprises cells that differentiate into endodermal lineages, cells that differentiate into mesodermal lineages, and cells that differentiate into ectodermal lineages upon exposure to appropriate induction signals.

Embodiment 42. The composition of any one of the preceding embodiments, wherein the composition further comprises a synthetic buffer.

Embodiment 43. The composition of any one of the preceding embodiments, wherein the composition further comprises a pharmaceutically-acceptable excipient.

Embodiment 44. The composition of any one of the preceding embodiments, wherein the composition does not contain serum.

Embodiment 45. The composition of any one of the preceding embodiments, wherein the composition is in a unit dosage form.

Embodiment 46. The composition of any one of the preceding embodiments, wherein the composition further comprises a culture medium that supports proliferation of the amniotic fluid stem cells.

Embodiment 47. A method of generating a clonal population of amniotic fluid stem cells, comprising: (a) isolating a single CD90 positive amniotic fluid stem cell from a plurality of cells based on expression of CD90; and (b) expanding the single CD90 positive amniotic fluid stem cell to generate the clonal population of amniotic fluid stem cells.

Embodiment 48. The method of embodiment 47, wherein the isolating comprises fluorescent activated cell sorting.

Embodiment 49. The method of embodiment 47, wherein the isolating comprises magnetic activated cell sorting.

Embodiment 50. A method of generating a population of amniotic fluid stem cells, comprising: (a) sorting a population of cells comprising amniotic fluid stem cells to obtain an enriched population of amniotic fluid stem cells comprising at least 90% CD90 positive cells; (b) expanding the enriched population of amniotic fluid stem cells in culture to obtain an expanded population of amniotic fluid stem cells; (c) sorting the expanded population of amniotic fluid stem cells to obtain a second enriched population of amniotic fluid stem cells comprising at least 90% CD90 positive cells; and (d) expanding the second enriched population of amniotic fluid stem cells in culture to obtain a second expanded population of amniotic fluid stem cells.

Embodiment 51. The method of any one of embodiments 47-50, wherein the expanding is conducted in medium that lacks serum.

Embodiment 52. The method of any one of embodiments 47-51, wherein the expanding comprises incubating in a culture medium that supports proliferation of the amniotic fluid stem cells.

Embodiment 53. The method of any one of embodiments 47-52, wherein the expanding comprises incubating in culture medium within a compartment that is coated by a thin layer of agarose.

Embodiment 54. A method of treating a condition in a subject in need thereof, comprising administering to the subject the composition of any one of embodiments 1-46.

Embodiment 55. The method of embodiment 54, wherein the condition is neonatal encephalopathy.

Embodiment 56. The method of embodiment 54, wherein the condition is traumatic brain injury.

Embodiment 57. The method of embodiment 54, wherein the condition is ischemia.

Embodiment 58. The method of embodiment 54, wherein the condition is type 2 diabetes mellitus.

Embodiment 59. The method of embodiment 54, wherein the condition is type 1 diabetes mellitus.

Embodiment 60. The method of embodiment 54, wherein the condition is cardiovascular disease.

Embodiment 61. The method of embodiment 54, wherein the condition is a progressive neurodegenerative disease.

Embodiment 62. The method of embodiment 54, wherein the condition is Alzheimer's disease.

Embodiment 63. The method of embodiment 54, wherein the condition is Huntington's disease.

Embodiment 64. The method of embodiment 54, wherein the condition is amyotrophic lateral sclerosis.

Embodiment 65. The method of embodiment 54, wherein the condition is multiple sclerosis.

Embodiment 66. The method of embodiment 54, wherein the condition is Parkinson's disease.

Embodiment 67. The method of embodiment 54, wherein the condition is multiple system atrophy.

Embodiment 68. The method of embodiment 54, wherein the condition is muscular dystrophy.

Embodiment 69. The method of embodiment 54, wherein the condition is a peripheral nerve injury.

Embodiment 70. The method of embodiment 54, wherein the condition is a peripheral muscle injury.

Embodiment 71. The method of embodiment 54, wherein the condition is a wound.

Embodiment 72. The method of embodiment 54, wherein the condition is an ulcer.

Embodiment 73. The method of embodiment 54, wherein the condition is a diabetic ulcer.

Embodiment 74. The method of embodiment 54, wherein the condition is a burn.

Embodiment 75. The method of embodiment 54, wherein the condition is arthritis.

Embodiment 76. The method any one of embodiments 54-75, wherein the method increases regeneration of a tissue in the subject.

Embodiment 77. The method of any one of embodiments 54-76, wherein the method increases regeneration of an organ in the subject.

Embodiment 78. The method of any one of embodiments 54-77, wherein the amniotic fluid stem cells are autologous to the subject.

Embodiment 79. The method of any one of embodiments 54-77, wherein the amniotic fluid stem cells are from a donor that is fully HLA-matched to the subject.

Embodiment 80. The method of any one of embodiments 54-77, wherein the amniotic fluid stem cells are from a donor that is partially HLA-matched to the subject.

Embodiment 81. The method of any one of embodiments 54-77, wherein the amniotic fluid stem cells are from a donor that is haploidentical to the subject.

Embodiment 82. The method of any one of embodiments 54-81, wherein the administering is intravenous.

Embodiment 83. The method of any one of embodiments 54-81, wherein administering is topical.

Embodiment 84. The method of any one of embodiments 54-81, wherein the administering is intradermal.

Embodiment 85. The method of any one of embodiments 54-81, wherein the administering is sub-mucosal.

Embodiment 86. The method of any one of embodiments 54-81, wherein the administering is intramuscular.

Embodiment 87. The method of any one of embodiments 54-81, wherein the administering is parenteral.

Embodiment 88. The method of any one of embodiments 54-81, wherein the administering is intrathecal.

Embodiment 89. The method of any one of embodiments 54-81, wherein the administering is by injection.

Embodiment 90. The method of any one of embodiments 54-81, wherein the administering is by infusion.

Embodiment 91. The method of any one of embodiments 54-90, wherein the amniotic fluid stem cells are administered at a dose of at least about 1×10\hat{ }5 cells.

Embodiment 92. The method of any one of embodiments 54-91, wherein the amniotic fluid stem cells are administered once.

Embodiment 93. The method of any one of embodiments 54-91, wherein the amniotic fluid stem cells are administered twice.

Embodiment 94. A method of determining an effect of an agent on stem cells, comprising contacting a population of cells with the agent, wherein the population of cells comprises amniotic fluid stem cells, and determining the effect of the agent on the amniotic fluid stem cells, wherein at least 65% of the cells in the population of cells are CD90 positive, wherein the population of cells comprises at least 50 cells.

Embodiment 95. A method of determining an effect of an agent on stem cells, comprising: (a) isolating a single CD90 positive amniotic fluid stem cell from a plurality of cells; (b) expanding the single CD90 positive amniotic fluid stem cell to generate a clonal population of amniotic fluid stem cells; (c) contacting the clonal population of amniotic fluid stem cells with the agent; and (d) determining the effect of the agent on the clonal population of amniotic fluid stem cells.

Embodiment 96. A method of determining an effect of an agent on stem cells, comprising: (a) sorting a population of cells comprising amniotic fluid stem cells to obtain an enriched population of amniotic fluid stem cells comprising at least 90% CD90 positive cells; (b) expanding the enriched population of amniotic fluid stem cells in culture to obtain an expanded population of amniotic fluid stem cells; (c) sorting the expanded population of amniotic fluid stem cells to obtain a second enriched population of amniotic fluid stem cells comprising at least 90% CD90 positive cells; (d) expanding the second enriched population of amniotic fluid stem cells in culture to obtain a second expanded population of amniotic fluid stem cells; (e) contacting the second expanded population of amniotic fluid stem cells with the agent; and (f) determining the effect of the agent on the second expanded population of amniotic fluid stem cells.

Embodiment 97. The method of any one of embodiments 94-96, wherein the agent is a drug.

Embodiment 98. The method of any one of embodiments 94-97, wherein the agent is a candidate teratogen.

Embodiment 99. The method of any one of embodiments 94-98, wherein the amniotic fluid stem cells comprise a dose-responsive reporter gene driven by an inducible promoter and the determining the effect of the agent on the clonal population of amniotic fluid stem cells comprises measuring an amount of the reporter gene that is expressed after the contacting.

Embodiment 100. The method of any one of embodiments 94-99, wherein the determining the effect of the agent on the clonal population of amniotic fluid stem cells comprises measuring expression of a marker associated with ectodermal differentiation after the contacting.

Embodiment 101. The method of any one of embodiments 94-100, wherein the determining the effect of the agent on the clonal population of amniotic fluid stem cells comprises measuring expression of a marker associated with mesodermal differentiation after the contacting.

Embodiment 102. The method of any one of embodiments 94-101, wherein the determining the effect of the agent on the clonal population of amniotic fluid stem cells comprises measuring expression of a marker associated with endodermal differentiation after the contacting.

Embodiment 103. The method of any one of embodiments 94-102 wherein the determining the effect of the agent on the clonal population of amniotic fluid stem cells comprises measuring expression of a marker associated with neuronal differentiation after the contacting.

Embodiment 104. The method of any one of embodiments 94-103, wherein the determining the effect of the agent on the clonal population of amniotic fluid stem cells comprises measuring expression of a marker associated with chondrocyte differentiation after the contacting.

Embodiment 105. The method of any one of embodiments 94-104, wherein the determining the effect of the agent on the clonal population of amniotic fluid stem cells comprises measuring expression of a marker associated with osteocyte differentiation after the contacting.

### EXAMPLES

### Example 1: Generation of a Clonal Population of Amniotic Fluid Stem Cells

Amniotic fluid samples were collected from 10 pregnant human subjects at second semester amniocentesis. The amniotic fluid samples (2-5 ml for each sample) were diluted with PBS, and then spun by centrifuge at 300 g (4° C.) for 5 minutes. The supernatant was discarded, and pelleted cells were resuspended in culture medium and transferred to 60 mm or 100 mm tissue culture dishes (Nunc Thermo Scientific, Hampton, N.H.). The dishes were incubated at 37° C. with 5% humidified CO₂ using complete AFSC medium containing MEM-alpha GlutaMAX (Life Technologies, Grand Island, N.Y.), 20% Chang medium D (Irvine Scientific, Santa Ana, Calif.), 15% embryonic stem cell-qualified fetal bovine serum (ES-FBS) (Life Technologies, Grand Island, N.Y.), 100 μg/mL normocin (InvivoGen, San Diego, Calif.). Cells were grown to ˜70% of confluence in a 37° C. humidified incubator with 5% CO₂. Absence of mycoplasma in the culture was periodically verified with the Plasmotest kit (InVivogen San Diego). Cells were detached from plates using Accutase (MP Biomedicals, Irvine, Calif.), and 30 samples were FACS sorted for cells that were CD90 positive, SSEA4 positive, and Tra-1-60 positive. Aliquots at least 90% CD90 positive were then used to select a single cell for further passaging in 96 well plates. Tissue quality, round bottom 96-well plates (BD Falcon, Franklin Lakes, N.J.) were coated with thin layer of 0.8% low melting agarose (Sigma-Aldrich, St. Louis, Mo.) dissolved in alpha-MEM-GlutaMAX by filling the wells with the melted agarose and then removing the fluid and allowing the remaining film of agarose to solidify during a brief incubation (˜5 minutes) at 4° C. Cells were seeded at a concentration of 1 cell in 100 μl to each coated well. The plates were incubated in 37° C. incubator with 5% CO₂ for 2 days. Spheroids thus obtained were transferred into receiving wells of a 24-well flat-bottomed tissue culture plate (BD Falcon, Franklin Lakes, N.J.) using wide bore pipette tips. After 2-3 passages, the clones were sorted again for CD90 positive cells. The culturing and FACS sorting for CD90 positive cells was repeated until clones were identified that exhibited consistent expression of CD90, with over 95% CD90 positive cells. SSEA4 and Tra 1-60 expression levels were observed to decrease to lower levels and remained consistent after the cycles of culturing and sorting. After the cultures remained stable, the four most consistent and viable cultures were selected for differentiation studies and further characterization by FACS for other surface markers and nuclear transcription factors. Cells were identified that expressed other stem cell markers, including Oct-4, Sox-2, Nanog, Rex-1, CD117, CD15, CD44, CD29, CD9, CD73 and CD133.

Respective differentiation media were added to each well and the plates were incubated at 37° C. in a 5% CO₂ atmosphere for 2-3 weeks. Control cultures were maintained in complete AFSC medium. After 2-3 weeks in culture, the two selected clones maintained in complete AFSC medium were consistently 99% CD90 positive, 27% SSEA4 positive, and 5-10% Tra-1-60 positive (after 5 passages). The other stem cell markers Oct-4, Sox-2, Nanog, Rex-1, CD117, 15, 44, 29, 9, 73 and 133 were expressed as well.

### Example 2: In Vitro Differentiation of Clonal Amniotic Fluid Stem Cells

In vitro studies are conducted to assess the differentiation potential of amniotic fluid stem cell clones (e.g., clones generated in Example 1). The cells can be treated with test agents (e.g., chemicals, drugs, etc.) to determine the effect of the agents on proliferation and differentiation of the stem cell clones. Illustrative, non-limiting examples of differentiation assays are provided below. Illustrative, non-limiting examples of test agents include valproic acid (VPA, a known teratogen), dexamethasone (DEX, promotes fetal maturation), and MgSO₄ (administered in pregnancy for seizure prophylaxis and fetal neuroprotection).

A. In Vitro Neural Differentiation

STEMDIFF™ Neural Induction medium (Stemcell Technologies, Vancouver, Canada) is used to differentiate the disclosed human amniotic fluid clonal stem cells toward a neuronal lineage. Spheroids are transferred into STEMDIFF Neural Induction medium supplemented with 10 mM Y-27632 ROCK inhibitor (Stem-Gent, Cambridge, Mass.), five spheroids per well, and differentiated for 21 days under the following conditions: unexposed to test agent, single exposure to test agent (various concentrations), and continuous exposure to test agent (various concentrations). Uninduced controls are included. The spheroids are fixed with 3.5% buffered formalin (Thermo Fisher Scientific, Hampton, N.H.) and incubated with 1:100 dilutions of the following monoclonal antibodies: mouse-anti-human-Nestin-AlexaFluor647 (BD Pharmingen), mouse anti-human B-tubulin III-AlexaFluor488 (BD Pharmingen) and mounted with VECTASHIELD Mounting Medium with DAPI (Vector Labs, Burlingame, Calif.), or mouse anti-human glial fibrillary acidic protein (GFAP)-AlexaFluor488 (BD Pharmingen) and VECTASHIELD Mounting Medium with DAPI (Vector Laboratories, Burlingham, Calif.). Spheroids are visualized under fluorescence microscopy with a Nikon Eclipse TE2000-E (Nikon Instruments, Inc.) inverted microscope, and images are acquired with Nikon NIS-Elements Imaging Software BR 3.10. Images are analyzed and quantitated using ImageJ 1.47v (National Institutes of Health, Bethesda, Md.) to determine whether the amniotic fluid stem cells differentiate toward a neuronal lineage, and whether the test agents (e.g., VPA, DEX, and MgSO₄) affect proliferation and differentiation of the human amniotic fluid clonal stem cells.

B. In Vitro Osteocyte Differentiation

Nonhematopoietic OSTEODIFF™ Medium (Miltenyi Biotech, Cambridge, Mass.) is used to differentiate homogeneous human amniotic fluid clonal stem cells to osteocytes; 4.5×10⁴ spheroids are used in each assay (1 assay=1 well of a 6-well tissue culture plate) (BD Falcon). Differentiation is carried out for 10 days with medium changes every 2-3 days. The spheroids are then transferred to osteocyte induction medium and incubated for 2 weeks. Differentiation is performed with samples unexposed to test agent, single exposure to test agent (various concentrations), and continuous exposure to test agent (various concentrations). Uninduced controls are included. Spheroids are either fixed with cold methanol and stained with SIGMA FAST BCIP/NBT (Sigma-Aldrich) for alkaline phosphatase-producing cells, or fixed with 3.7% buffered formalin for 30 minutes at room temperature and stained with 2% Alizarin Red solution pH 4.3 (Sigma-Aldrich) for 45 minutes in the dark at room temperature. Spheroids are visualized under bright-field and phase-contrast microscopy as above to determine whether the amniotic fluid stem cells differentiate toward an osteocyte lineage, and whether the test agents (e.g., VPA, DEX, and MgSO₄) affect proliferation and differentiation of the human amniotic fluid clonal stem cells. Osteogenic induction patterns are then studied under the same conditions with Ab83369 (Abcam, Cambridge, Mass.), a quantitative alkaline phosphatase enzymatic colorimetric assay, to determine whether the amniotic fluid stem cells differentiate toward an osteocyte lineage, and whether the test agents (e.g., VPA, DEX, and MgSO₄) affect proliferation and differentiation of the human amniotic fluid clonal stem cells.

C. In Vitro Chondrocyte Differentiation

Nonhematopoietic OSTEODIFF™ Medium (Miltenyi Biotech, Cambridge, Mass.) is used to differentiate homogeneous human amniotic fluid clonal stem cells to chondrocytes. The spheroids are transferred to a 4-well chamber slide, five spheroids per well, under the following medium conditions: unexposed to test agent, single exposure to test agent (various concentrations), and continuous exposure to test agent (various concentrations). Uninduced controls are included. The spheroids are incubated for 21 days, with medium changes every 2-3 days. The spheroids are fixed in 3.5% buffered formalin (Thermo Fisher Scientific) and incubated with 1:100 dilutions of the primary monoclonal antibody, mouse anti-human Aggrecan antibody (Invitrogen, Carlsbad, Calif.), and 1:200 dilution of the secondary AlexaFluor594 goat anti-Rabbit IgG (H+L) antibody (Invitrogen). Coverslips are mounted with VECTASHIELD Mounting Medium with DAPI (Vector Laboratories). Spheroids are visualized under fluorescence microscopy with a Nikon Eclipse TE2000-E (Nikon Instruments, Inc.) inverted microscope and images are acquired with Nikon NIS-Elements Imaging Software BR 3.10. Images are analyzed and quantitated using ImageJ 1.47v (National Institutes of Health) to determine whether the amniotic fluid stem cells differentiate toward a chondrocyte lineage, and whether the test agents (e.g., VPA, DEX, and MgSO₄) affect proliferation and differentiation of the human amniotic fluid clonal stem cells.

D. Transcriptomic Responses of Amniotic Fluid Stem Cells to Test Agents

Samples from each subject are treated with test agents of interest, e.g., valproic acid (VPA, a known teratogen), dexamethasone (DEX, promotes fetal maturation), or MgSO₄ (administered in pregnancy for seizure prophylaxis and fetal neuroprotection). Untreated controls are included. After treatment, cells are harvested and RNA collected using an RNeasy Mini Kit (Qiagen, Valencia, Calif.). A NanoDrop Spectrophotometer (ND200) (Thermo Fisher Scientific, Springfield Township, N.J.) is used to determine RNA concentration and purity. Gene expression analysis is performed using the Affymetrix method of expression analysis, with principal component analysis (PCA) for gene expression. The Affymetrix Expression Console software is used to extract expression data from each microarray chip using the robust multiarray average algorithm. Data are imported into the R statistical programming environment. The R/Bio-conductor package, Limma (Linear Models for Microarray Data, version 3.22.3), is used to assess the quality of the data and to analyze for differential expression after control and low expressing probes are filtered from the data set. The data are analyzed to determine whether the test agents (e.g., VPA, DEX, and MgSO₄) affect gene expression of the human amniotic fluid clonal stem cells.

E. Statistical Analysis

The cells of each sample are separately analyzed by ImageJ for chondrogenic and neural differentiation studies. For osteogenic induction, samples are analyzed using an alkaline phosphatase assay kit (Ab83369, Abcam). At least five cells are used for each sample and each analysis is done in triplicate. Mean and standard deviation are calculated for results of induction for each assay. As the number of cells varies for each sample, an average of averages is calculated for reporting each condition. For genomic studies, enrichment score ≥2, fold change ≥2.0, and P value <0.01 are considered significant. Bonferroni and Benjamini corrections are applied and compared with the uncorrected data.

F. Transcriptomic Analysis

PCA is employed to determine significant differences in gene expression between treated and untreated cell cultures. Database for Annotation, Visualization, and Integrated Discovery (DAVID) is utilized for cluster analysis to determine pertinent affected functions due to exposure to the agents studied. GOTERM FAT library is chosen to reflect gene ontology and biological processes. Ingenuity Pathway Analysis (IPA) (IPA®; Qiagen, Redwood City, Calif.) is utilized to analyze data identified by Affymetrix analysis for both VPA and DEX. Canonical pathways, network functions, and disease and pathway heat maps are generated and analyzed. All function results are coded to reflect either upregulation or downregulation and to reflect z-score. The data are analyzed to determine whether the test agents (e.g., VPA, DEX, and MgSO₄) affect gene expression of the human amniotic fluid clonal stem cells.

### Example 3: An Amniotic Fluid Stem Cell Clone Promotes Allogeneic Skin Engraftment

Amniotic fluid stem cells (AFSC) have immunomodulatory and anti-inflammatory properties. An experimental study was performed to determine whether application of AFSCs in a collagen matrix on transplanted skin facilitated tolerance of skin grafts.

Six to eight week old black C57BL/6 mice and six to eight week old black Balb/c mice were used as donors and recipients of skin grafts. Each group of mice included five animals. For each group, three animals were experimental and two animals were used as vehicle control. The animals were anesthetized and the animals' backs were shaved and then a depilatory agent was applied to remove all hair. A 1.5-cm-diameter circular wound was inflicted on each animal, sterilized and coated with PURACOL®. White mice skin graft was applied to the 1.5 cm wound square template on each animal.

7.2×10⁶ CD90+ amniotic fluid stem cloned cells were prepared, stained with 1,1′-Dioctadecyl-3,3,3′,3′-Tetramethylindocarbocyanine Perchlorate (DiI) and applied to each graft side of the experimental animals. Placebo was applied to each graft side of the vehicle control animals. In a first set of mice the cloned cells were applied topically, and in a second set of mice the cloned cells were injected subcutaneously around each graft.

The treated mice were observed at post-operative day (POD) 5 and at POD 7 for signs of rejection, such as edema and/or erythema. At POD 7, tissues were collected from one control and one experimental mouse for each group in each set. The tissues were formalin-fixed and paraffin-embedded, stained with hematoxylin and eosin, and optical coherence tomography (OCT) imaging was performed. The tissues were further stained with CD11B or F4/80, C4d, and CD68 to observe acute rejection, and photographed every POD.

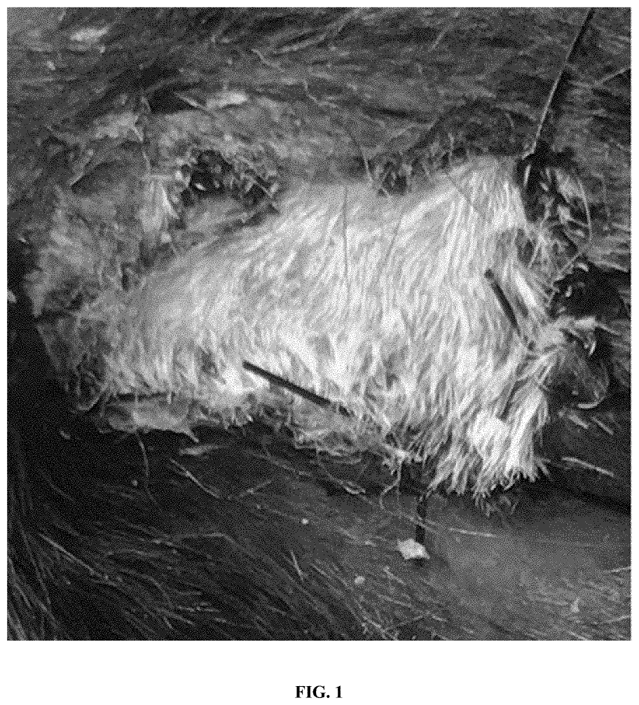

Vehicle control mice rejected their grafts by POD 10 or POD 11. In contrast, experimental mice treated with the cloned cells showed no signs of rejection before day 14, and most clone-treated grafts lasted 21 days, 10 days longer than vehicle control mice. These results demonstrated that the cloned amniotic fluid stem cells induced significant graft tolerance. The experimental black mice treated with the cloned cells also showed a healthy white graft and significant hair growth on the treated grafts, indicating enhanced vascularization. See FIG. 1.

### Example 4: Treatment of Ischemia with an Amniotic Fluid Stem Cell Clone

A pharmaceutical composition comprising human amniotic fluid clonal stem cells of the disclosure is injected into the ischemic hind limbs of mice. An assay is conducted to determine whether the amniotic fluid stem cells differentiate into vascular-like structures and express endothelial-specific genes and proteins. An assay is conducted to determine whether the amniotic fluid stem cells promote recruitment and activity of endogenous cells that promote vascularization. An assay is conducted to determine the engraftment rate. An assay is conducted to determine whether the amniotic fluid stem cells augment blood perfusion and capillary density, indicating neovascularization.

### Example 5: In Vitro and In Vivo Regeneration of Skeletal Muscle

An amniotic fluid stem cell clone of the disclosure is co-cultured in vitro with C2C12 myoblasts. Differentiation analysis is conducted to determine whether the amniotic fluid stem cells differentiate into skeletal myogenic cells, and express skeletal myogenic cell-specific markers such as Desmin, Troponin I (Tn I) and α-Actinin.

A pharmaceutical composition comprising amniotic fluid stem cells of the disclosure is injected into cardiotoxin-injured and X-ray-irradiated tibialis anterior (TA) muscles of NOD/SCID mice. An assay is conducted to determine whether the cells differentiate into myogenic precursor cells and fuse with host myofibers.

### Example 6: Biodegradable Scaffolds for Tissue Repair

Amniotic fluid stem cells of the disclosure are proliferated in a chondrogenic medium containing transforming growth factor-beta 2 (TGF-beta2) and insulin-like growth factor-1 (IGF-1) for 6-12 weeks. The cells are then seeded onto a biodegradable polyglycolic acid scaffold and maintained in the same chondrogenic medium within a rotating bioreactor for 10-15 weeks to produce a tendon graft. The engineered scaffold is surgically inserted into a subject (e.g., a lamb) for diaphragmatic hernia repair. Examination is conducted to determine whether the amniotic fluid stem cells show evidence of chondrocyte differentiation and regeneration of cartilage.

### Example 7: Treatment of Alzheimer's Disease with Amniotic Fluid Stem Cells

The safety and efficacy of an amniotic fluid stem cell cone of the disclosure is assessed in subjects with moderate-to-severe Alzheimer's disease. A pharmaceutical composition comprising amniotic fluid stem cells is injected into subjects' brains in two initial doses on days 1 and 15. This experimental regimen is compared to an analogous regimen in which a pharmaceutical composition containing placebo is administered in place of the disclosed amniotic fluid clonal stem cells. 10 subjects are included in each of the two groups. Subjects are monitored for disease activity and symptoms, such as confusion and forgetfulness, over a period of one year.

At the end of the one year period, subjects are evaluated to determine whether subjects treated with the disclosed pharmaceutical composition show an improvement of symptoms and indicators of Alzheimer's disease over the control.

### Example 8: Generation and Characterization of a Clonal Population of Amniotic Fluid Stem Cells

Amniotic Fluid Stem Cells and Cloning

A midtrimester amniotic fluid sample was obtained from a patient with a clinically indicated amniocentesis. The patient gave written informed consent for the sample to be used for research purposes. The sample had a normal karyotype.

A sample of amniotic fluid stem cells was taken from cryopreservation, thawed and placed back into culture in complete AFSC medium containing MEM-alpha GlutaMAX (Life Technologies, Grand Island, N.Y.), 20% Chang medium D (Irvine Scientific, Santa Ana, Calif.), 15% embryonic stem cell-qualified fetal bovine serum (ES-FBS) (Life Technologies, Grand Island, N.Y.), and 100 ug/mL normocin (InvivoGen, San Diego, Calif.). Cells were grown to ˜70% of confluence in a 37° C. humidified incubator with 5% CO₂. Cells were detached from 100 mm tissue culture dishes (Nunc Thermo Scientific, Hampton, N.H.) using Accutase (MP Biomedicals, Irvine, Calif.). Cells were washed in 1× Dulbecco's Phosphate-buffered saline (DPBS) and 5% ES-FBS, stained 1:1000 dilution with anti-human CD9O-PerCP-Cy5.5 (BD Pharmingen, San Jose, Calif.), and co-stained with propidium iodide (PI) for fluorescent activated single cell sorting using a MoFlo XDP sorter. Single CD90 PerCP-Cy5.5 positive and PI negative cells were sorted for into wells of a flat-bottom 96-well plate filled with complete AFSC medium, at one cell per well. Five plates were sorted, sealed with parafilm and returned to incubation for 14 days. Plates were screened for clonal growth using a Nikon Phase Contrast 2 ELWD 0.3 inverted scope. Confluent wells were marked. Medium removed was removed from these wells, and the cells were washed with 1×DPBS, detached with Accutase, and transferred to 6-well plates for expansion. Cells were subsequently passaged to larger 60 and 100 mm tissue culture plates (Nunc Thermo Scientific, Hampton, N.H.) and cultured until clones were observed to undergo senescence. After initial culture, an aliquot was cryopreserved at every passage for subsequent use.

Flow Cytometry

Clones were reanalyzed for CD90 expression starting with passage 3 and also analyzed with the following antibodies for stem cell surface markers: mouse monoclonal anti-human SSEA4-APC (BD Pharmingen, San Jose, Calif.), mouse monoclonal anti-human CD73-PE-Cy7 (BD Pharmingen, San Jose, Calif.), mouse monoclonal anti-human CD44-FITC (BD Pharmingen, San Jose, Calif.), mouse monoclonal anti-human CD29-PE (BD Pharmingen, San Jose, Calif.), and mouse monoclonal anti-human CD105-BV-421 (BD Pharmingen, San Jose, Calif.). Staining was performed at 4° C. protected from light, and stained cells analyzed on a BD LSRII flow cytometer (BD Biosciences, San Jose, Calif.). Analysis was performed with WinList™ software (Verity Software House, Topsham, Me.). Flow cytometry reanalysis was performed for each clone every other passage until reaching senescence.

Spheroid Production

Previous work has demonstrated the speed and efficacy of differentiation when AFSC are grown as three dimensional cell cultures, which can be referred to as spheroids. Spheroids were created by using V-bottom 96-well plates (Corning, Corning, N.Y.). The plates were first coated with a thin layer of 0.8% low-melting agarose (Sigma Aldrich, St. Louis, Mo.) dissolved in MEM-alpha GlutaMAX. After filling the wells with melted agarose, the fluid was removed and the remaining agarose film solidified for five minutes at 4° C. A concentration of 10,000 cells in 100 uL were placed in each well and the plate then incubated for two days at 37° C. with 5% CO₂. The spheroids were then transferred to receiving wells of flat-bottomed tissue culture vessels and differentiation media added depending on which type of differentiation was intended. Five to ten spheroids were used per induction, the procedure repeated a second time and the results averaged.

Neural Differentiation

To differentiate AFSC toward neural linage, STEMdiff™ Neural Induction medium (SNIM, Stemcell Technologies Inc., Vancouver, Canada) was used according to the manufacturer's protocol. The neural induction medium was supplemented with 10 μM Y-27632 ROCK inhibitor. The spheroids were transferred into the medium, five spheroids per well of Poly-L-ornithine (Sigma-Aldrich, St. Louis, Mo.) and laminin (Sigma-Aldrich, St. Louis, Mo.) treated chamber slides (Millipore, Billerica, Mass.), and were cultured for fourteen days. The spheroids were fixed with 3.5% buffered formalin, permeabilized in 0.1% Triton X-100, 10% FBS and 1×DPBS before incubated with 1:100 dilutions of various monoclonal antibodies. These antibodies included those for Nestin-AlexaFluor 647 (BD Pharmingen, San Jose, Calif.), B-tubulin III-AlexaFluor 488 (BD Pharmingen, San Jose, Calif.) and glial fibrillary acidic protein (GFAP)-AlexaFluor 488 (BD Pharmingen, San Jose, Calif.). The spheroids were then visualized with fluorescent microscopy using a Nikon Eclipse TE2000-E (Nikon Instruments Inc., Melville, N.Y.) inverted microscope and images were acquired with Nikon NIS-Elements Imaging Software BR 3.10. The average mean intensity of the fluorescence of the images was analyzed and quantitated using ImageJ 1.47v (National Institutes of Health, Bethesda, Md.).

Chondrocyte Differentiation

Differentiation of AFSC into chondrocytes was performed using Non-Hematopoetic ChondroDiff Medium according to the manufacturer's protocol over 24 days with medium changes occurring every 2-3 days. Chondrocyte nodules were then fixed in 3.7% neutral buffered formalin and immunostained for aggrecan to detect cartilage production using monoclonal mouse anti-human aggrecan. Spheroids were permeabilized in 0.1% Triton X-100, 10% FBS and 1×DPBS, before incubation with Aggrecan that was diluted 1:100 in phosphate buffered saline (overnight at room temperature). A 1:200 dilution of an AlexaFluor594 goat anti-Rabbit IgG (H+L) secondary antibody (Invitrogen, Carlsbad, Calif.) was incubated at room temperature overnight and visualized with fluorescent microscopy using a Nikon Eclipse TE2000-E (Nikon Instruments Inc., Melville, N.Y.) inverted microscope. Images were acquired with Nikon NIS-Elements Imaging Software BR 3.10. The average mean intensity of the fluorescence of the images was analyzed and quantitated using ImageJ 1.47v (National Institutes of Health, Bethesda, Md.).

Definitive Endoderm Differentiation

Differentiation of AFSC into definitive endoderm was performed using StemXVivo Endoderm Kit (R&D Systems, Minneapolis, Minn.). Spheroids were transferred into the medium and incubated according to the manufacturer's protocol. The spheroids were fixed with 3.5% buffered formalin, permeabilized in 0.1% Triton X-100, 10% FBS and 1×DPBS before incubation with 1:100 dilutions of monoclonal antibodies of mouse-anti-human-CXCR4-PE (eBioscience, San Diego, Calif.) and mouse-anti-human-SOX17-AlexaFluor488 (BD Pharmingen, San Jose, Calif.). The spheroids were then visualized with fluorescent microscopy using a Nikon Eclipse TE2000-E (Nikon Instruments Inc., Melville, N.Y.) inverted microscope and images were acquired with Nikon NIS-Elements Imaging Software BR 3.10. The average mean intensity of the fluorescence of the images was analyzed and quantitated using ImageJ 1.47v (National Institutes of Health, Bethesda, Md.).

Gene Expression Analysis

Gene expression analysis was performed on clone 115 to determine presence of genes associated with stem cell properties, such as OCT4. mRNA was extracted from cultured amniotic fluid stem cells from clone 115 using an RNeasy Mini Kit (Qiagen Valencia, Calif.). Concentration of mRNA was determined by A260 using a NanoDrop Lite spectrophotometer (Thermo Scientific, Hampton, N.H.). Residual genomic DNA was digested and mRNA was reverse transcribed to cDNA using iScript™ gDNA Clear cDNA Synthesis Kit (Bio-Rad Laboratories, Hercules, Calif.). Primers for OCT4, SOX2, REX01, NANOG, and PROM1 were obtained from Bio-Rad Laboratories PrimerPCR™. Real-time PCR was carried out using iQ SYBR Green Supermix (Bio-Rad Laboratories, Hercules, Calif.) on a Bio-Rad iCycler, which was also used for analysis. Each reaction used 100 ng of cDNA. HPRT1 and G6PDX, widely expressed housekeeping genes, served as positive controls. Negative controls were performed with sterile water as substrate. For each reaction, 40 cycles were performed. Melting curves were obtained to verify proper primer function. HPRT1 and G6PDX expression were used to normalize the data. Each reaction was performed in triplicate.

Real time PCR results were reported in delta Ct (cycle threshold) values, which subtract measured threshold value from the maximum 40 cycles, utilizing a logarithmic scale. Lower delta Ct value is associated with lower level of expression.

Results

Clones exhibited cell doubling times ranging from 25 to 47 hours. Of the 480 clones that were sorted into five 96-well plates, 130 clones were transferred to 6-well plates for expansion. Of those 130 clones, 7 clones that made it past passage 3 were expanded for further studies.

To characterize the clones that highly expressed CD90, cells from subsequent passages were subjected to flow cytometry. In addition to confirming CD90 expression, expression of other stem cell markers was evaluated, including SSEA4, CD29, CD44, CD73, and CD105. The cell surface marker expression data are summarized in Tables 1-6. Of the initial batch of clones, only clone 115 was able to be kept in culture up to passage 10 before reaching senescence. Clone 115 exhibited consistent marker expression through passage 10 except for a gradual loss of SSEA4 and a moderate decline in CD105.

TABLE 1
percentage of cells positive for CD90
for each clone from passages P3 to P10.
TABLE 1

| Clone | P3 | P4 | P5 | P6 | P7 | P8 | P9 | P10 |
|---|---|---|---|---|---|---|---|---|
| 1 | 49 | 23 | 52 | | | | | |
| 88 | 99 | 98 | 93 | | | | | |
| 93 | 99 | 95 | 76 | | | | | |
| 94 | 100 | | 98 | | | | | |
| 97 | 99 | | 99 | | | | | |
| 102 | 100 | | 99 | 83 | | | | |
| 115 | 96 | | 95 | 95 | 97 | 98 | 100 | 99 |

TABLE 2
percentage of cells positive for SSEA4
for each clone from passages P3 to P10.
TABLE 2

| Clone | P3 | P4 | P5 | P6 | P7 | P8 | P9 | P10 |
|---|---|---|---|---|---|---|---|---|
| 1 | 11 | 3 | 0.5 | | | | | |
| 88 | 65 | 27 | 3 | | | | | |
| 93 | 38 | 11 | 1 | | | | | |
| 94 | 68 | | 27 | | | | | |
| 97 | 82 | | 56 | | | | | |
| 102 | 63 | | 27 | 1 | | | | |
| 115 | 58 | | 19 | 9 | 15 | 9 | 0 | 0.5 |

TABLE 3
percentage of cells positive for CD29
for each clone from indicated passages.
TABLE 3

| Clone | P5 | P6 | P7 | P8 | P9 | P10 |
|---|---|---|---|---|---|---|
| 1 | 100 | | | | | |
| 88 | 100 | | | | | |
| 93 | 100 | | | | | |
| 102 | | 100 | | | | |
| 115 | | 99 | 98 | 99 | 100 | 99 |

TABLE 4
percentage of cells positive for CD44
for each clone from indicated passages.
TABLE 4

| Clone | P5 | P6 | P7 | P8 | P9 | P10 |
|---|---|---|---|---|---|---|
| 1 | 100 | | | | | |
| 88 | 94 | | | | | |
| 93 | 68 | | | | | |
| 102 | | 99 | | | | |
| 115 | | 95 | 99 | 99 | 100 | 100 |

TABLE 5
percentage of cells positive for CD73
for each clone from indicated passages.
TABLE 5

| Clone | P5 | P6 | P7 | P8 | P9 | P10 |
|---|---|---|---|---|---|---|
| 1 | 100 | | | | | |
| 88 | 100 | | | | | |
| 93 | 100 | | | | | |
| 102 | | 100 | | | | |
| 115 | | 99 | 99 | 99 | 100 | 100 |

TABLE 6
percentage of cells positive for CD105
for each clone from indicated passages.
TABLE 6

| Clone | P5 | P6 | P7 | P8 | P9 | P10 |
|---|---|---|---|---|---|---|
| 1 | 13 | | | | | |
| 88 | 18 | | | | | |
| 93 | 32 | | | | | |
| 102 | | 18 | | | | |
| 115 | | 73 | 53 | 30 | 28 | 12 |

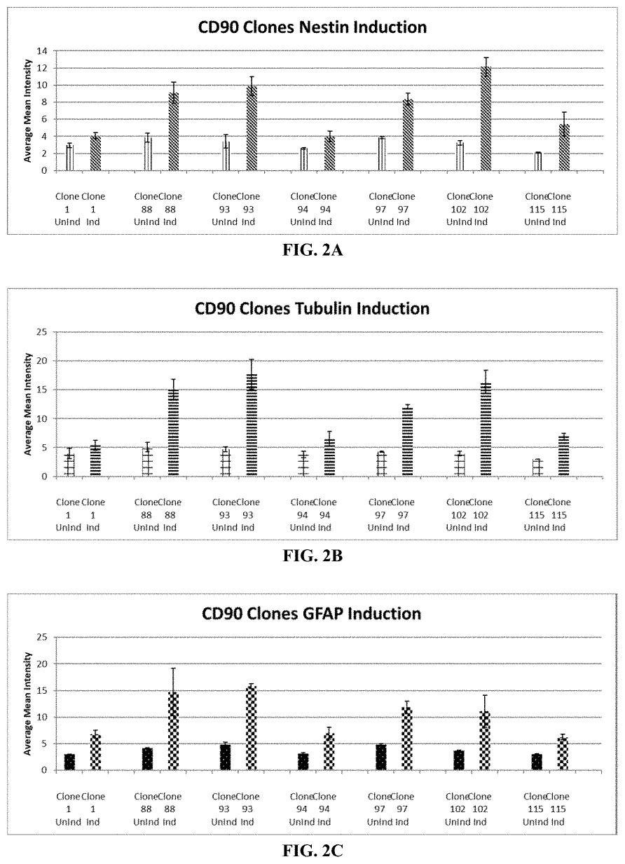

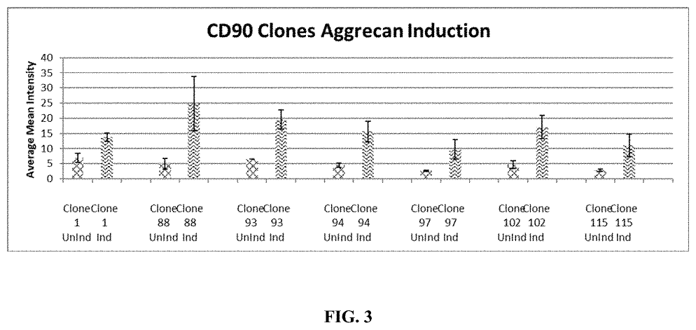

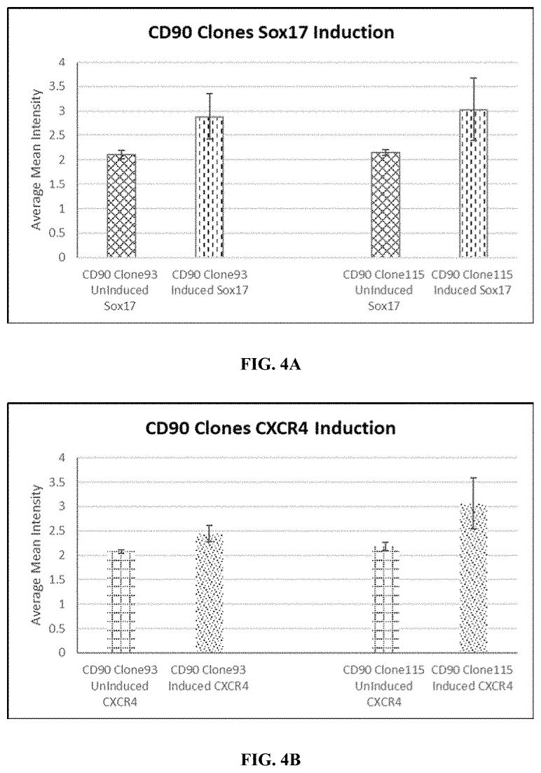

The clones reached sufficient cell numbers to form three dimensional cell culture spheroids by passage 5. Spheroids were formed to induce towards neural and chondrogenic lineages. The CD90 clones differentiate towards a neural lineage, expressing Nestin (FIG. 2A), Beta-Tubulin III (FIG. 2B), and glial fibrillary acidic protein (GFAP) (FIG. 2C). The CD90 clones are able to differentiate towards a chondrogenic lineage expressing Aggrecan (FIG. 3). CD90 clones are also able to induce towards definitive endoderm expressing SOX17 (FIG. 4A) and CXCR4 (FIG. 4B).

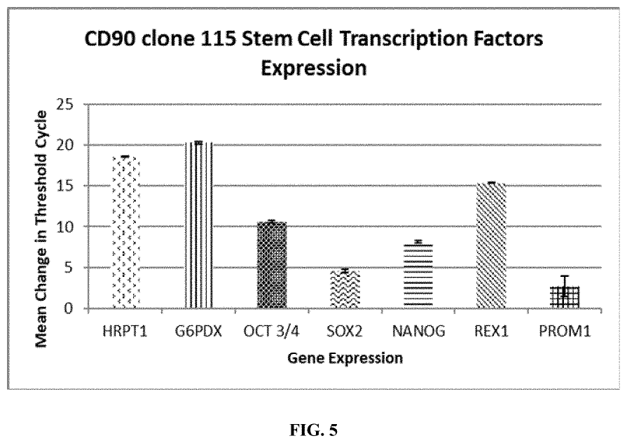

Given the capability of CD90 clone 115 to remain in cell culture up to passage 10 without undergoing senescence, and the capability of the clone to differentiate down neural, chondrogenic and definitive endoderm lineages, this clone was further characterized for expression of stem cell transcription factors and other relevant genes by quantitative real time PCR. Expression of the following genes was examined: OCT3/4, SOX2, NANOG, REX1, and PROM1. These genes were found to be expressed when compared to the housekeeping control genes of HRPT1 and G6PDX (FIG. 5).

These results demonstrate that a clonal pluripotent stem cell line can be generated from amniotic fluid stem cells.

1-105. (canceled)

106. A composition comprising a population of amniotic fluid stem cells, wherein at least 90% of cells in the population of amniotic fluid stem cells are CD90 positive, wherein at least 90% of cells in the population of amniotic fluid stem cells remain CD90 positive after two weeks in culture.

107. The composition of claim 106, wherein the population of amniotic fluid stem cells has been prepared by a process comprising:
(a) isolating a single CD90 positive amniotic fluid stem cell from a plurality of amniotic fluid stem cells based on expression of CD90; and
(b) expanding the single CD90 positive amniotic fluid stem cell to generate the population of amniotic fluid stem cells.

108. The composition of claim 106, wherein the population of amniotic fluid stem cells has been prepared by a process comprising:
(a) sorting an initial population of cells to obtain an enriched population of amniotic fluid stem cells comprising at least 90% CD90 positive cells;
(b) expanding the enriched population of amniotic fluid stem cells in culture to obtain an expanded population of amniotic fluid stem cells;
(c) sorting the expanded population of amniotic fluid stem cells to obtain a second enriched population of amniotic fluid stem cells comprising at least 90% CD90 positive cells; and
(d) expanding the second enriched population of amniotic fluid stem cells in culture to obtain a second expanded population of amniotic fluid stem cells.

109. The composition of claim 106, wherein at least 3% of cells in the population of amniotic fluid stem cells are TRA-1-60 positive.

110. The composition of claim 106, wherein at least 10% of cells in the population of amniotic fluid stem cells are SSEA4 positive.

111. The composition of claim 106, wherein at least 95% of cells in the population of amniotic fluid stem cells are CD90 positive.

112. The composition of claim 106, wherein the population of amniotic fluid stem cells comprises at least 1×10⁵ cells.

113. The composition of claim 106, wherein cells in the population of amniotic fluid stem cells are progeny of amniotic fluid stem cells harvested from a pregnant human during the pregnant human's second trimester of pregnancy or later and cultured in vitro.

114. The composition of claim 106, wherein the population of amniotic fluid stem cells comprises cells that have been cultured in vitro for at least 7 days.

115. The composition of claim 106, wherein the population of amniotic fluid stem cells comprises cells that have divided at least 10 times in vitro.

116. The composition of claim 106, wherein the population of amniotic fluid stem cells comprises cells that differentiate towards a neuronal lineage as determined by an increase in nestin, beta tubulin III, and glial fibrillary acidic protein expression after incubation in neural induction medium.

117. The composition of claim 106, wherein the population of amniotic fluid stem cells comprises cells that differentiate into chondrocytes as determined by an increase in aggrecan after incubation in chondrocyte induction medium.

118. The composition of claim 106, wherein the population of amniotic fluid stem cells comprises cells that differentiate into endodermal lineages as determined by an increase in SOX17 and CXCR4 expression after incubation in endodermal induction medium.

119. The composition of claim 106, wherein after two weeks in culture in vitro, the population of amniotic fluid stem cells comprises cells that differentiate into endodermal lineages, cells that differentiate into mesodermal lineages, and cells that differentiate into ectodermal lineages upon exposure to appropriate induction signals.

120. The composition of claim 106, wherein the composition further comprises a synthetic buffer.

121. The composition of claim 106, wherein the composition further comprises a pharmaceutically-acceptable excipient.

122. The composition of claim 106, wherein the composition does not contain serum.

123. The composition of claim 106, wherein the composition is in a unit dosage form.

124. A method of generating a clonal population of amniotic fluid stem cells, comprising:
(a) isolating a single CD90 positive amniotic fluid stem cell from a plurality of cells based on expression of CD90; and
(b) expanding the single CD90 positive amniotic fluid stem cell to generate the clonal population of amniotic fluid stem cells.

125. A method of generating a population of amniotic fluid stem cells, comprising:
(a) sorting a population of cells comprising amniotic fluid stem cells to obtain an enriched population of amniotic fluid stem cells comprising at least 90% CD90 positive cells;
(b) expanding the enriched population of amniotic fluid stem cells in culture to obtain an expanded population of amniotic fluid stem cells;
(c) sorting the expanded population of amniotic fluid stem cells to obtain a second enriched population of amniotic fluid stem cells comprising at least 90% CD90 positive cells; and
(d) expanding the second enriched population of amniotic fluid stem cells in culture to obtain a second expanded population of amniotic fluid stem cells.
